(19) United States
(12) Patent Application Publication
(10) Pub. No.: US 2025/0367125 A1
(43) Pub. Date: Dec. 4, 2025

(54) ORAL POLYPEPTIDE COMPOSITION

(71) Applicant: Shenzhen Oralead Pharma Co., LTD., Shenzhen (CN)

(72) Inventors: Banghao YUAN, Shenzhen (CN); Yusheng Sui, Shenzhen (CN); Liu Dou, Shenzhen (CN)

(73) Assignee: Shenzhen Oralead Pharma Co., LTD., Shenzhen (CN)

(21) Appl. No.: 19/194,059

(22) Filed: Apr. 30, 2025

Related U.S. Application Data
(63) Continuation of application No. PCT/CN2025/084442, filed on Mar. 24, 2025.

(30) Foreign Application Priority Data
Jun. 3, 2024 (CN) 202410710353.4
Sep. 24, 2024 (CN) 202411335782.4

(51) Int. Cl. A61K 9/48; A61K 9/00; A61K 9/20; A61K 31/166; A61K 31/198; A61K 31/20; A61K 38/26

(52) U.S. Cl. CPC A61K 9/4825; A61K 9/0053; A61K 9/2013; A61K 9/2054; A61K 9/2059; A61K 9/4866; A61K 31/166; A61K 31/198; A61K 31/20; A61K 38/26

(57) ABSTRACT

An oral polypeptide composition for improving the gastrointestinal absorption of polypeptide drugs is provided. The oral polypeptide composition includes polypeptide molecules, medium-chain fatty acids (MCFA) and their salts, amino acids and N-(8-(2-hydroxybenzoyl) amino) caprylic acid (NAC) and its salts. The oral polypeptide composition can effectively increase the plasma drug concentration of the polypeptide and improve the oral bioavailability of the polypeptide.

### CROSS REFERENCE TO THE RELATED APPLICATIONS

This application is a continuation application of International Application No. PCT/CN2025/084442, filed on Mar. 24, 2025, which is based upon and claims priority to Chinese Patent Application No. 202410710353.4, filed on Jun. 3, 2024, and Chinese Patent Application No. 202411335782.4, filed on Sep. 24, 2024, the entire contents of which are incorporated herein by reference.

### TECHNICAL FIELD

The present disclosure belongs to the field of biomedicine, and specifically relates to an oral polypeptide composition.

### BACKGROUND

Polypeptide drugs are a class of biological macromolecules formed by short-chain amino acids connected through peptide bonds, and usually contain 10 to 50 amino acids. They are an important branch in the current drug research and development field. Compared with small-molecule drugs, polypeptide drugs can precisely regulate specific biological processes without affecting other physiological functions. Compared with many macromolecular biopharmaceuticals, polypeptides usually have lower immunogenicity because their smaller molecular structures and more natural-like peptide characteristics make them less likely to trigger a strong immune response. Compared with complex biopharmaceuticals, the synthesis process of polypeptide drugs is simpler and more controllable, which makes the production process more efficient and cost-effective. At the same time, natural polypeptides can also be modified in various means to enhance their efficacy, improve pharmacokinetic characteristics or reduce side effects.

Currently, the main administration method of polypeptide drugs is by injection. Injection is an invasive administration method via the skin, which may cause patients' pain and discomfort. In addition, injection usually requires the operation of medical professionals or by the patients themselves, which increases the complexity and inconvenience of drug administration. Injection may also lead to complications such as injection-site infections and local tissue damage, especially when the operation is improper or the same site is repeatedly injected. Due to the pain and inconvenience brought by injection, patients may be unwilling to receive injections regularly as prescribed by doctors, affecting the treatment effect. Therefore, it is very necessary to develop oral dosage forms of polypeptide drugs.

Although oral polypeptide drugs have obvious advantages in terms of convenience and acceptability, the scientific and technological problems encountered in their development are still quite severe. These challenges are mainly manifested in the generally low bioavailability of polypeptide drugs, mainly due to stability and absorption barriers. Gastric acid and intestinal enzymes rapidly degrade polypeptides, significantly reducing their oral bioavailability. To achieve an effective treatment, it is necessary to ensure that a sufficient amount of the drug can stably pass through the digestive system and enter the blood circulation. In addition, due to the large volume and polar characteristics of polypeptide drug molecules, they are difficult to penetrate the intestinal mucosa, and even if the polypeptide can remain stable in the intestine, its absorption efficiency may still be low.

Rybelsus® (oral semaglutide) is the first approved oral glucagon-like peptide-1 (GLP-1) receptor agonist, which marks a major breakthrough in the field of polypeptide therapy. The success of Rybelsus® not only establishes a new pathway for semaglutide but also facilitates the development and commercialization of other potential oral polypeptide drugs. It demonstrates that with appropriate formulations and technologies, bioactive molecules traditionally administered by injection can be formulated for oral delivery.

Rybelsus® adopts the Eligen® technology developed by Emisphere Technologies. This technology uses special chemical carriers called “Eligen carriers” to achieve its effect. Rybelsus® uses sodium N-(8-(2-hydroxybenzoyl)amino) caprylate (SNAC) in the Eligen carriers. It increases the stability and absorption rate of semaglutide in the stomach by adjusting the pH value in the stomach and affecting the permeability of the gastric mucosa. However, clinical data show that the bioavailability of Rybelsus® is only about 0.8%. Bioavailability refers to the proportion of a drug that enters the systemic circulation from the administration site. For drugs, low bioavailability means that only a small part of the administered dose finally reaches the blood circulation and takes effect. To compensate for low bioavailability, higher drug doses are required, increasing both costs and the risk of side effects. Low bioavailability often results in significant interindividual variability, leading to unstable or unpredictable therapeutic effects. If frequent administration is required to compensate for the low bioavailability, it may reduce the patient's treatment compliance, resulting in poor treatment effects and improper disease management.

In conclusion, as a revolutionary polypeptide therapeutic drug, although Rybelsus® brings the convenience of oral treatment to patients, there is still the problem of low bioavailability that needs to be urgently solved.

### SUMMARY

The purpose of the present disclosure is to provide an oral polypeptide composition.

Another purpose of the present disclosure is to provide a method for preparing an oral polypeptide composition.

Another purpose of the present disclosure is to provide a use of an oral polypeptide composition.

Another purpose of the present disclosure is to provide an oral polypeptide composition comprising a polypeptide targeting a GLP-1 receptor and its use.

One aspect of the present disclosure provides an oral polypeptide composition comprising: a polypeptide molecule, one or more medium-chain fatty acids or salts thereof, one or more amino acids, and N-(8-(2-hydroxybenzoyl) amino) caprylic acid (NAC) or salts thereof.

In some embodiments, the polypeptide is a pharmaceutically acceptable peptide.

In some embodiments, the polypeptide molecule is a compound comprising multiple amino acids or comprising at least one peptide bond, or a pharmaceutically acceptable salt thereof.

In some embodiments, the molecular weight of the polypeptide molecule is about 0.1 kDa to 20 kDa, preferably 0.1 kDa to 15 kDa, preferably 0.1 kDa to 10 kDa, preferably 0.1 kDa to 9.0 kDa, preferably 0.1 kDa to 5.0 kDa, preferably 0.1 kDa to 2.0 kDa; preferably 0.5 kDa to 20 kDa, preferably 0.5 kDa to 15 kDa, preferably 0.5 kDa to 10 kDa, preferably 0.5 kDa to 9.0 kDa, preferably 0.5 kDa to 5.0 kDa, preferably 0.5 kDa to 2.0 kDa; preferably 1.0 kDa to 20 kDa, preferably 1.0 kDa to 15 kDa, preferably 1.0 kDa to 10 kDa, preferably 1.0 kDa to 9.0 kDa, preferably 1.0 kDa to 5.0 kDa, preferably 1.0 kDa to 2.0 kDa; preferably 5.0 kDa to 20 kDa, preferably 5.0 kDa to 15 kDa, preferably 5.0 kDa to 10 kDa, preferably 5.0 kDa to 9.0 kDa; preferably 10.0 kDa to 20 kDa, preferably 10.0 kDa to 15 kDa.

In some embodiments, the molecular weight of the polypeptide molecule is about 0.1 kDa, 0.5 kDa, 1 kDa, 1.5 kDa, 2 kDa, 2.5 kDa, 3 kDa, 3.5 kDa, 4 kDa, 4.5 kDa, 5 kDa, 5.5 kDa, 6 kDa, 6.5 kDa, 7 kDa, 7.5 kDa, 8 kDa, 8.5 kDa, 9 kDa, 9.5 kDa, 10 kDa, 15 kDa or 20 kDa.

In some embodiments, the polypeptide molecule comprises at least one linear structure and/or at least one cyclic structure.

In some embodiments, the polypeptide molecule is a peptide with a linear structure, a peptide with a cyclic structure, or a peptide with both a linear structure and a cyclic structure.

In some embodiments, the polypeptidemolecule is selected from a modified peptide, a derived peptide, or a peptidomimetic peptide.

In some embodiments, the polypeptide molecule is selected from GLP-1, GLP-1 analogs, GLP-1 receptor agonists, semaglutide, liraglutide, tirzepatide, exenatide, exenatide-4, lixisenatide, taspoglutide, langlenatide, GLP-1 (7-37), GLP-1 (7-36) NH₂, GLP-2, GLP-2 agonists or analogs, somatostatin or analogs, parathyroid hormone (PTH) fragments or analogs, or pharmaceutically acceptable salts thereof.

In some embodiments, the pharmaceutically acceptable salt are water-soluble salts and water-insoluble salts.

In some embodiments, the GLP-1 analog is selected from acylated GLP-1 analog, diacylated GLP-1 analog, and long-acting albumin-bound fatty acid-derivatized GLP-1 analog.

In some embodiments, the GLP-1 receptor agonist is selected from a dual agonist of GLP-1 receptor/glucagon receptor (GCGR), a dual agonist of GLP-1 receptor/glucose-dependent insulinotropic polypeptide (GIP) receptor, a dual agonist of GLP-1 receptor/amylin receptor, and a triple agonist of GLP-1 receptor/GIP receptor/GCGR.

In some embodiments, the GLP-2 agonist or analog includes teduglutide and elsiglutide.

In some embodiments, the somatostatin or analog comprises octreotide.

In some embodiments, the parathyroid hormone fragment or analog comprises teriparatide.

In some embodiments, the polypeptide molecule is selected from the semaglutide, teriparatide, liraglutide, octreotide or tirzepatide.

In some embodiments, the polypeptide molecule is selected from the semaglutide, teriparatide, octreotide or tirzepatide.

In some embodiments, the polypeptide molecule is selected from the semaglutide, liraglutide and tirzepatide.

In some embodiments, the medium-chain fatty acid is a fatty acid with a carbon chain length of 6 to 12 carbon atoms, and its structural formula is CH₃(CH₂)ₙCOOH, wherein n is greater than or equal to 4 and less than or equal to 10.

In some embodiments, the medium-chain fatty acid is selected from one or more of caproic acid, enanthic acid, caprylic acid, pelargonic acid, capric acid, undecanoic acid and lauric acid.

In some embodiments, the medium-chain fatty acid salt is selected from one or more of sodium salt, potassium salt and ammonium salt.

In some embodiments, the medium-chain fatty acid salt is sodium caprate.

In some embodiments, the amino acid is an amino acid comprising nitrogen in the side chain.

In some embodiments, the amino acid comprising nitrogen in the side chain is selected from at least one of lysine, arginine, asparagine, glutamine, tryptophan, ornithine, citrulline, pyrrolysine and histidine.

In some embodiments, the amino acid comprising nitrogen in the side chain is selected from at least one of citrulline and arginine.

In some embodiments, the N-(8-(2-hydroxybenzoyl) amino) caprylic acid (NAC) salt is selected from one or more of N-(8-(2-hydroxybenzoyl) amino) caprylic acid sodium salt, N-(8-(2-hydroxybenzoyl) amino) caprylic acid potassium salt and N-(8-(2-hydroxybenzoyl) amino) caprylic acid ammonium salt.

In some embodiments, the salt of NAC is sodium N-(8-(2-hydroxybenzoyl) amino) caprylate (SNAC).

In some embodiments, the ratio of the polypeptide molecule, the medium-chain fatty acid or its salt, amino acid and N-(8-(2-hydroxybenzoyl) amino) caprylic acid (NAC) or its salt is (0.1-100):(20-1000):(1-200):(20-160).

In some embodiments, the site of action of the oral polypeptide composition is the gastrointestinal tract.

In some embodiments, the oral polypeptide composition can promote the stable absorption of polypeptide drug molecules in the gastrointestinal tract.

In some embodiments, the oral polypeptide composition has different microstructures.

In another aspect, the present disclosure provides an oral polypeptide formulation, which comprises the oral polypeptide composition described herein and pharmaceutically acceptable excipients.

In some embodiments, the oral polypeptide formulation is selected from tablets, capsules, capsule in capsules, micro-patch systems in capsules, lozenges, tablets, Ovules, solutions, emulsions, suspensions, syrups, ointments, powders and granules for reconstitution, dispersible powders and granules, pharmaceutically acceptable gums, chewable tablets, effervescent tablets and multi-granular dosage forms.

In some embodiments, the pharmaceutically acceptable excipients comprise at least one of fillers, glidants, granulation binders, lubricants and disintegrants.

In another aspect, the present disclosure provides a method for preventing and/or treating diseases, comprising administering the oral polypeptide composition to a subject in need; wherein the diseases comprise endocrine diseases, neurodegenerative diseases, vascular diseases, kidney diseases, fatty liver diseases, and cancer, polycystic ovary syndrome, bone metabolism and osteoporosis, knee osteoarthritis, sleep apnea syndrome, osteoarthritis, acromegaly, carcinoid syndrome, islet cell tumors, intestinal fistulas, esophageal varicose bleeding, and metabolic muscle diseases.

In some embodiments, the endocrine diseases comprise glucose metabolism disorders, diabetes, and obesity.

In some embodiments, the neurodegenerative diseases comprise Alzheimer's disease and other forms of dementia, Parkinson's disease, multiple sclerosis, and Huntington's disease.

In some embodiments, the cardiovascular diseases comprise atherosclerosis, hyper-coagulable state and hypo-coagulable state, coronary artery disease, and cerebrovascular events.

In some embodiments, the kidney diseases comprise renal failure.

In some embodiments, the fatty liver comprises non-alcoholic fatty liver.

Another aspect of the present disclosure provides an oral polypeptide composition that comprises: a polypeptide targeting the GLP-1 receptor, one or more amino acids, N-(8-[2-hydroxybenzoyl]-amino) caprylic acid (NAC) or a salt thereof and one or more medium-chain fatty acids or salts thereof.

In some embodiments, the polypeptide composition does not comprise nicotinamide, adenosine, adenine, guanine, cytosine, trigonelline and salicylamide.

In some embodiments, the polypeptide composition comprises a polypeptide targeting GLP-1 receptor, one or more amino acids, N-(8-[2-hydroxybenzoyl]-amino) caprylic acid (NAC) or a salt thereof and one or more medium-chain fatty acids or salts thereof; and the polypeptide composition does not comprise a coordination molecule, and the coordination molecule is a C₆₋₁₀ aromatic ring or C₅₋₁₀ heteroaromatic ring compound comprising a hydrophilic group.

In some embodiments, the coordination molecule is selected from nicotinamide, adenosine, adenine, guanine, cytosine, trigonelline and salicylamide.

In some embodiments, the composition comprises no more than about 55% w/w N-(8-[2-hydroxybenzoyl]-amino) caprylic acids (NAC) or salts thereof.

In some embodiments, the composition comprises no more than about 50% w/w N-(8-[2-hydroxybenzoyl]-amino) caprylic acids (NAC) or salts thereof.

In some embodiments, the composition comprises no more than about 45% w/w N-(8-[2-hydroxybenzoyl]-amino) caprylic acid (NAC) or salts thereof.

In some embodiments, the composition comprises no more than about 40% w/w N-(8-[2-hydroxybenzoyl]-amino) caprylic acid (NAC) or salts thereof.

In some embodiments, the composition comprises no more than about 35% w/w N-(8-[2-hydroxybenzoyl]-amino) caprylic acid (NAC) or salts thereof.

In some embodiments, the composition comprises about 49% w/w, 48% w/w, 47% w/w, 46% w/w, 45% w/w, 44% w/w, 43% w/w, 42% w/w, 41% w/w, 40% w/w, 39% w/w, 38% w/w, 37% w/w, 36% w/w or 35% w/w N-(8-[2-hydroxybenzoyl]-amino)caprylic acid (NAC) or salts thereof.

In some embodiments, the composition comprises no less than about 20% w/w N-(8-[2-hydroxybenzoyl]-amino) caprylic acid (NAC) or salts thereof.

In some embodiments, the composition comprises no less than about 25% w/w N-(8-[2-hydroxybenzoyl]-amino) caprylic acid (NAC) or salts thereof.

In some embodiments, the composition comprises no less than about 30% w/w N-(8-[2-hydroxybenzoyl]-amino) caprylic acid (NAC) or salts thereof.

In some embodiments, the composition comprises about 21% w/w, 22% w/w, 23% w/w, 24% w/w, 25% w/w, 26% w/w, 28% w/w, 29% w/w or 30% w/w N-(8-[2-hydroxybenzoyl]-amino) caprylic acid (NAC) or salts thereof.

In some embodiments, the composition comprises about 20-50% w/w N-(8-[2-hydroxybenzoyl]-amino) caprylic acid (NAC) or salts thereof.

In some embodiments, the composition comprises about 20-45% w/w N-(8-[2-hydroxybenzoyl]-amino) caprylic acid (NAC) or salts thereof.

In some embodiments, the composition comprises about 20-40% w/w N-(8-[2-hydroxybenzoyl]-amino) caprylic acid (NAC) or salts thereof.

In some embodiments, the composition comprises about 25-50% w/w N-(8-[2-hydroxybenzoyl]-amino) caprylic acid (NAC) or salts thereof.

In some embodiments, the composition comprises about 25-45% w/w N-(8-[2-hydroxybenzoyl]-amino) caprylic acid (NAC) or salts thereof.

In some embodiments, the composition comprises about 25-40% w/w N-(8-[2-hydroxybenzoyl]-amino) caprylic acid (NAC) or salts thereof.

In some embodiments, the composition comprises about 30-50% w/w N-(8-[2-hydroxybenzoyl]-amino) caprylic acid (NAC) or salts thereof.

In some embodiments, the composition comprises about 30-45% w/w N-(8-[2-hydroxybenzoyl]-amino) caprylic acid (NAC) or salts thereof.

In some embodiments, the composition comprises about 30-40% w/w N-(8-[2-hydroxybenzoyl]-amino) caprylic acid (NAC) or salts thereof.

In some embodiments, the composition comprises about 30-35% w/w N-(8-[2-hydroxybenzoyl]-amino) caprylic acid (NAC) or salts thereof.

In some embodiments, the composition comprises no more than about 20% w/w amino acid.

In some embodiments, the composition comprises no more than about 18% w/w amino acid.

In some embodiments, the composition comprises no more than about 15% w/w amino acid.

In some embodiments, the composition comprises no more than about 15% w/w amino acid.

In some embodiments, the composition comprises no more than about 19% w/w, 18% w/w, 17% w/w, 16% w/w, 15% w/w, 14% w/w, 13% w/w or 12% w/w amino acid.

In some embodiments, the composition comprises no less than about 5% w/w amino acid.

In some embodiments, the composition comprises no less than about 7% w/w amino acid.

In some embodiments, the composition comprises no less than about 8% w/w amino acid.

In some embodiments, the composition comprises no less than about 9% w/w amino acid.

In some embodiments, the composition comprises no less than about 10% w/w amino acid.

In some embodiments, the composition comprises no less than about 5% w/w, 5.5% w/w, 6% w/w, 7.5% w/w, 7.8% w/w, 8.3% w/w, 8.5% w/w, 8.8% w/w, 9% w/w, 9.2% w/w, 9.5% w/w or 10% w/w amino acid.

In some embodiments, the composition comprises about 5-20% w/w amino acid.

In some embodiments, the composition comprises about 7-20% w/w amino acid.

In some embodiments, the composition comprises about 7-18% w/w amino acid.

In some embodiments, the composition comprises about 7-15% w/w amino acid.

In some embodiments, the composition comprises about 8-20% w/w amino acid.

In some embodiments, the composition comprises about 8-18% w/w amino acid.

In some embodiments, the composition comprises about 8-15% w/w amino acid.

In some embodiments, the composition comprises about 9-20% w/w amino acid.

In some embodiments, the composition comprises about 10-20% w/w amino acid.

In some embodiments, the composition comprises about 9-15% w/w amino acid.

In some embodiments, the composition comprises about 10-15% w/w amino acid.

In some embodiments, the composition comprises no more than about 40% w/w medium-chain fatty acids or salts thereof.

In some embodiments, the composition comprises no more than about 35% w/w medium-chain fatty acids or salts thereof.

In some embodiments, the composition comprises no more than about 39% w/w, 38% w/w, 37% w/w, 36% w/w or 35% w/w medium-chain fatty acids or salts thereof.

In some embodiments, the composition comprises no less than 10% w/w medium-chain fatty acids or salts thereof.

In some embodiments, the composition comprises no less than 15% w/w medium-chain fatty acids or salts thereof.

In some embodiments, the composition comprises no less than 20% w/w medium-chain fatty acids or salts thereof.

In some embodiments, the composition comprises no less than 30% w/w medium-chain fatty acids or salts thereof.

In some embodiments, the composition comprises no less than 10% w/w, 12% w/w, 14% w/w, 15% w/w, 16% w/w, 18% w/w, 21% w/w, 22% w/w, 23% w/w, 24% w/w, 25% w/w, 26% w/w, 28% w/w, 29% w/w or 30% w/w medium-chain fatty acids or salts thereof.

In some embodiments, the composition comprises about 10-40% w/w medium-chain fatty acids or salts thereof.

In some embodiments, the composition comprises about 15-40% w/w medium-chain fatty acids or salts thereof.

In some embodiments, the composition comprises about 15-35% w/w medium-chain fatty acids or salts thereof.

In some embodiments, the composition comprises about 20-40% w/w medium-chain fatty acids or salts thereof.

In some embodiments, the composition comprises about 25-35% w/w medium-chain fatty acids or salts thereof.

In some embodiments, the composition comprises about 25-40% w/w medium-chain fatty acids or salts thereof.

In some embodiments, the composition comprises about 30-40% w/w medium-chain fatty acids or salts thereof.

In some embodiments, the composition comprises about 30-35% w/w medium-chain fatty acids or salts thereof.

In some embodiments, the composition comprises no more than about 15% w/w polypeptide targeting the GLP-1 receptor.

In some embodiments, the composition comprises no more than about 10% w/w polypeptide targeting the GLP-1 receptor.

In some embodiments, the composition comprises about 0.1-15% w/w polypeptide targeting the GLP-1 receptor.

In some embodiments, the composition comprises about 0.5-10% w/w polypeptide targeting the GLP-1 receptor.

In some embodiments, the composition comprises about 0.8-10% w/w polypeptide targeting the GLP-1 receptor.

In some embodiments, the composition comprises about 1.0-7.0% w/w polypeptide targeting the GLP-1 receptor.

In some embodiments, the composition comprises about 0.2% w/w, 0.8% w/w, 0.9% w/w, 1.0% w/w, 1.5% w/w, 1.6% w/w, 1.7% w/w, 1.8% w/w, 3.3% w/w, 3.4% w/w, 6.9% w/w, 7.0% w/w, 8.0% w/w, 9.0% w/w, 10.0% w/w polypeptide targeting the GLP-1 receptor.

In some embodiments, the total content of the amino acids, N-(8-[2-hydroxybenzoyl]-amino) caprylic acid (NAC) or a salt thereof and medium-chain fatty acids or salts thereof in the composition is no more than about 90% w/w.

In some embodiments, the total content of the amino acids, N-(8-[2-hydroxybenzoyl]-amino) caprylic acid (NAC) or a salt thereof and medium-chain fatty acids or salts thereof in the composition is no more than about 85% w/w.

In some embodiments, the total content of the amino acids, N-(8-[2-hydroxybenzoyl]-amino) caprylic acid (NAC) or a salt thereof and medium-chain fatty acids or salts thereof in the composition is no more than about 80% w/w.

In some embodiments, the total content of the amino acids, N-(8-[2-hydroxybenzoyl]-amino) caprylic acid (NAC) or a salt thereof and medium-chain fatty acids or salts thereof in the composition is no less than about 50% w/w.

In some embodiments, the total content of the amino acids, N-(8-[2-hydroxybenzoyl]-amino) caprylic acid (NAC) or a salt thereof and medium-chain fatty acids or salts thereof in the composition is no less than about 60% w/w.

In some embodiments, the total content of the amino acids, N-(8-[2-hydroxybenzoyl]-amino) caprylic acid (NAC) or a salt thereof and medium-chain fatty acids or salts thereof in the composition is no less than about 70% w/w.

In some embodiments, the total content of the amino acids, N-(8-[2-hydroxybenzoyl]-amino) caprylic acid (NAC) or a salt thereof and medium-chain fatty acids or salts thereof in the composition is about 50-90% w/w.

In some embodiments, the total content of the amino acids, N-(8-[2-hydroxybenzoyl]-amino) caprylic acid (NAC) or a salt thereof and medium-chain fatty acids or salts thereof in the composition is about 50-80% W/W.

In some embodiments, the total content of the amino acids, N-(8-[2-hydroxybenzoyl]-amino) caprylic acid (NAC) or a salt thereof and medium-chain fatty acids or salts thereof in the composition is about 60-80% w/w.

In some embodiments, the total content of the amino acids, N-(8-[2-hydroxybenzoyl]-amino) caprylic acid (NAC) or a salt thereof and medium-chain fatty acids or salts thereof in the composition is about 70-80% w/w.

In some embodiments, the total content of the amino acids, N-(8-[2-hydroxybenzoyl]-amino) caprylic acid (NAC) or a salt thereof and medium-chain fatty acids or salts thereof in the composition is about 60% w/w, 62% w/w, 63% w/w, 64% w/w, 65% w/w, 67% w/w, 68% w/w, 70% w/w, 71% w/w, 72% w/w, 73% w/w, 74% w/w, 75% w/w, 75.5% w/w, 75.8% w/w, 75.9% w/w, 77% w/w, 79% w/w or 80% w/w.

In some embodiments, the mass percentage content of the amino acids does not exceed that of N-(8-[2-hydroxybenzoyl]-amino) caprylic acid (NAC) or its salt.

In some embodiments, the mass percentage content of the amino acids does not exceed that of medium-chain fatty acids or their salts.

In some embodiments, the mass percentage content of the medium-chain fatty acids or their salts does not exceed that of N-(8-[2-hydroxybenzoyl]-amino) caprylic acid (NAC) or its salt.

In some embodiments, the mass ratio of the amino acid, N-(8-[2-hydroxybenzoyl]-amino) caprylic acid (NAC) or its salt and the medium-chain fatty acids or their salts is about (1-3):3:(1.5-6).

In some embodiments, the mass ratio of the N-(8-[2-hydroxybenzoyl]-amino) caprylic acid (NAC) or its salt to the medium-chain fatty acids or their salts is about 1:(0.5-2), such as 1:1, 2:1, 1.5:1, 1:1.5.

In some embodiments, the mass ratio of the amino acid to the N-(8-[2-hydroxybenzoyl]-amino) caprylic acid (NAC) or its salt is about (1-2):3, such as 1:3, 2:3, 1.5:3.

In some embodiments, the mass ratio of the amino acid, N-(8-[2-hydroxybenzoyl]-amino) caprylic acid (NAC) or its salt, and medium-chain fatty acids or their salts is about 1:(2.5-3.5):(2.5-3.5).

In some embodiments, the mass ratio of the amino acid, N-(8-[2-hydroxybenzoyl]-amino) caprylic acid (NAC) or its salt, and medium-chain fatty acids or their salts is about 1:(2.6-3.4):(2.6-3.4).

In some embodiments, the mass ratio of the amino acid, N-(8-[2-hydroxybenzoyl]-amino) caprylic acid (NAC) or its salt, and medium-chain fatty acids or their salts is about 1:(2.7-3.3):(2.7-3.3).

In some embodiments, the mass ratio of the amino acid, N-(8-[2-hydroxybenzoyl]-amino) caprylic acid (NAC) or its salt, and medium-chain fatty acids or their salts is about 1:(2.8-3.2):(2.8-3.2).

In some embodiments, the mass ratio of the amino acid, N-(8-[2-hydroxybenzoyl]-amino) caprylic acid (NAC) or its salt, and medium-chain fatty acids or their salts is about 1:(2.9-3.1):(2.9-3.1).

In some embodiments, the mass ratio of the amino acid, N-(8-[2-hydroxybenzoyl]-amino) caprylic acid (NAC) or its salt, and medium-chain fatty acids or their salt is about 1:3:3.

In some embodiments, the mass ratio of N-(8-[2-hydroxybenzoyl]-amino) caprylic acid (NAC) or its salt and the medium-chain fatty acids or their salts is about 1:1.

In some embodiments, the polypeptide targeting GLP-1 receptor is a GLP-1 receptor agonist.

In some embodiments, the polypeptide targeting GLP-1 receptor is semaglutide.

In some embodiments, the composition comprises: semaglutide, no more than about 20% w/w amino acids, no more than about 55% w/w N-(8-[2-hydroxybenzoyl]-amino) caprylic acid (NAC) or its salt, and medium-chain fatty acids or their salts.

In some embodiments, the composition comprises: semaglutide, no more than about 20% w/w amino acids, no more than about 55% w/w N-(8-[2-hydroxybenzoyl]-amino) caprylic acid (NAC) or its salt, and no less than about 10% w/w medium-chain fatty acids or their salts.

In some embodiments, the composition comprises: semaglutide, no more than about 20% w/w amino acids, no more than about 55% w/w N-(8-[2-hydroxybenzoyl]-amino) caprylic acid (NAC) or its salt, and no less than about 15% w/w medium-chain fatty acids or their salts.

In some embodiments, the composition comprises: semaglutide, no more than about 20% w/w amino acids, no more than about 50% w/w N-(8-[2-hydroxybenzoyl]-amino) caprylic acid (NAC) or its salt, and no less than about 10% w/w medium-chain fatty acids or their salts.

In some embodiments, the composition comprises: semaglutide, about 5-20% w/w amino acids, about 20-55% w/w N-(8-[2-hydroxybenzoyl]-amino) caprylic acid (NAC) or its salt, and no less than about 10% w/w medium-chain fatty acids or their salts.

In some embodiments, the composition comprises: semaglutide, about 10-20% w/w amino acids, about 20-55% w/w N-(8-[2-hydroxybenzoyl]-amino) caprylic acid (NAC) or its salt, and no less than about 10% w/w medium-chain fatty acids or their salts.

In some embodiments, the composition comprises: semaglutide, about 10-20% w/w amino acids, about 20-55% w/w N-(8-[2-hydroxybenzoyl]-amino) caprylic acid (NAC) or its salt, and no less than about 15% w/w medium-chain fatty acids or their salts.

In some embodiments, the composition comprises: semaglutide, one or more amino acids, N-(8-[2-hydroxybenzoyl]-amino)caprylic acid (NAC) or a salt thereof, and one or more medium-chain fatty acids or salts thereof; wherein the total content of the amino acids, N-(8-[2-hydroxybenzoyl]-amino) caprylic acid (NAC) or a salt thereof and medium-chain fatty acids or salts thereof in the composition is no more than about 90% w/w.

In some embodiments, the composition comprises: semaglutide, one or more amino acids, N-(8-[2-hydroxybenzoyl]-amino)caprylic acid (NAC) or a salt thereof, and one or more medium-chain fatty acids or salts thereof; wherein the total content of the amino acids, N-(8-[2-hydroxybenzoyl]-amino) caprylic acid (NAC) or a salt thereof and medium-chain fatty acids or salts thereof in the composition is about 50-90% w/w.

In some embodiments, the composition comprises: semaglutide, about 10-20% w/w amino acids, N-(8-[2-hydroxybenzoyl]-amino)caprylic acid (NAC) or a salt thereof, and one or more medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is no more than about 90% w/w.

In some embodiments, the composition comprises: semaglutide, about 5-20% w/w amino acids, N-(8-[2-hydroxybenzoyl]-amino)caprylic acid (NAC) or a salt thereof, and one or more medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is no less than about 50% w/w.

In some embodiments, the composition comprises: semaglutide, about 5-20% w/w amino acids, N-(8-[2-hydroxybenzoyl]-amino)caprylic acid (NAC) or a salt thereof, and one or more medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 50-90% w/w.

In some embodiments, the composition comprises: semaglutide, about 10-20% w/w amino acids, N-(8-[2-hydroxybenzoyl]-amino)caprylic acid (NAC) or a salt thereof, and one or more medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 50-90% w/w.

In some embodiments, the composition comprises: semaglutide, about 10-20% w/w amino acids, N-(8-[2-hydroxybenzoyl]-amino)caprylic acid (NAC) or a salt thereof, and one or more medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is no more than about 80% w/w.

In some embodiments, the composition comprises: semaglutide, about 10-20% w/w amino acids, about 20-55% N-(8-[2-hydroxybenzoyl]-amino)caprylic acid (NAC) or a salt thereof, and one or more medium-chain fatty acids or salts thereof; wherein the total content of the amino acids, N-(8-[2-hydroxybenzoyl]-amino) caprylic acid (NAC) or a salt thereof and medium-chain fatty acids or salts thereof in the composition is no more than about 80% w/w.

In some embodiments, the composition comprises: semaglutide, about 10-20% w/w amino acids, about 20-55% N-(8-[2-hydroxybenzoyl]-amino)caprylic acid (NAC) or a salt thereof, and no less than about 10% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is no more than about 90% w/w.

In some embodiments, the composition comprises: semaglutide, about 10-20% w/w amino acids, about 20-50% N-(8-[2-hydroxybenzoyl]-amino)caprylic acid (NAC) or a salt thereof, and no less than about 15% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is no more than about 90% w/w.

In some embodiments, the composition comprises: semaglutide, about 10-20% w/w amino acids, about 20-55% N-(8-[2-hydroxybenzoyl]-amino)caprylic acid (NAC) or a salt thereof, and no less than about 10% w/w medium-chain fatty acids or salts thereof; wherein the total content of the amino acids, N-(8-[2-hydroxybenzoyl]-amino) caprylic acid (NAC) or a salt thereof and medium-chain fatty acids or salts thereof in the composition is no more than about 80% w/w.

In some embodiments, the composition comprises: semaglutide, about 10-20% w/w amino acids, about 20-50% N-(8-[2-hydroxybenzoyl]-amino)caprylic acid (NAC) or a salt thereof, and no less than about 15% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is no more than about 80% w/w.

In some embodiments, the composition comprises: semaglutide, about 10-20% w/w amino acids, about 20-55% N-(8-[2-hydroxybenzoyl]-amino)caprylic acid (NAC) or a salt thereof, and no less than about 10% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w.

In some embodiments, the composition comprises: semaglutide, about 10-20% w/w amino acids, about 20-50% N-(8-[2-hydroxybenzoyl]-amino)caprylic acid (NAC) or a salt thereof, and no less than about 15% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w.

In some embodiments, the composition comprises: semaglutide, about 10-20% w/w amino acids, about 20-55% N-(8-[2-hydroxybenzoyl]-amino)caprylic acid (NAC) or a salt thereof, and about 10-40% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w.

In some embodiments, the composition comprises: semaglutide, about 10-20% w/w amino acids, about 20-50% N-(8-[2-hydroxybenzoyl]-amino)caprylic acid (NAC) or a salt thereof, and about 15-40% w/w medium-chain fatty acids or salts thereof; wherein the total content of the amino acids, N-(8-[2-hydroxybenzoyl]-amino) caprylic acid (NAC) or a salt thereof and medium-chain fatty acids or salts thereof in the composition is about 70-80% w/w.

In some embodiments, the composition comprises: semaglutide, about 10-20% w/w amino acids, about 20-50% N-(8-[2-hydroxybenzoyl]-amino)caprylic acid (NAC) or a salt thereof, and about 20-40% w/w medium-chain fatty acids or salts thereof; wherein the total content of the amino acids, N-(8-[2-hydroxybenzoyl]-amino) caprylic acid (NAC) or a salt thereof and medium-chain fatty acids or salts thereof in the composition is about 70-80% w/w.

In some embodiments, the composition comprises: semaglutide, about 10-20% w/w amino acids, about 20-55% N-(8-[2-hydroxybenzoyl]-amino)caprylic acid (NAC) or a salt thereof, and about 10-40% w/w medium-chain fatty acids or salts thereof; wherein the total content of the amino acids, N-(8-[2-hydroxybenzoyl]-amino) caprylic acid (NAC) or a salt thereof and medium-chain fatty acids or salts thereof in the composition is about 50-90% w/w; wherein the mass ratio of the N-(8-[2-hydroxybenzoyl]-amino) caprylic acid (NAC) or its salt to the medium-chain fatty acids or their salts is about 1:(0.5-2).

In some embodiments, the composition comprises: semaglutide, about 10-20% w/w amino acids, about 20-55% N-(8-[2-hydroxybenzoyl]-amino)caprylic acid (NAC) or a salt thereof, and about 10-40% w/w medium-chain fatty acids or salts thereof; wherein the total content of the amino acids, N-(8-[2-hydroxybenzoyl]-amino) caprylic acid (NAC) or a salt thereof and medium-chain fatty acids or salts thereof in the composition is about 70-80% w/w; wherein the mass ratio of the N-(8-[2-hydroxybenzoyl]-amino) caprylic acid (NAC) or its salt to the medium-chain fatty acids or their salts is about 1:(0.5-2).

In some embodiments, the composition comprises: semaglutide, about 10-20% w/w amino acids, about 20-50% N-(8-[2-hydroxybenzoyl]-amino)caprylic acid (NAC) or a salt thereof, and no less than about 15% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w; wherein the mass ratio of the N-(8-[2-hydroxybenzoyl]-amino) caprylic acid (NAC) or its salt to the medium-chain fatty acids or their salts is about 1:(0.5-2).

In some embodiments, the composition comprises: semaglutide, about 10-20% w/w amino acids, about 20-50% N-(8-[2-hydroxybenzoyl]-amino)caprylic acid (NAC) or a salt thereof, and no less than about 15% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w; wherein the mass ratio of the amino acid, N-(8-[2-hydroxybenzoyl]-amino) caprylic acid (NAC) or its salt, and medium-chain fatty acids or their salts is about (1-3):3:(1.5-6).

In some embodiments, the composition comprises: semaglutide, about 10-20% w/w amino acids, about 20-50% N-(8-[2-hydroxybenzoyl]-amino)caprylic acid (NAC) or a salt thereof, and no less than about 15% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w; wherein the mass ratio of the amino acid, N-(8-[2-hydroxybenzoyl]-amino) caprylic acid (NAC) or its salt, and medium-chain fatty acids or their salts is about 1:(2.5-3.5):(2.5-3.5).

In some embodiments, the composition comprises: no more than 10% w/w semaglutide, about 10-20% w/w amino acids, about 20-50% N-(8-[2-hydroxybenzoyl]-amino)caprylic acid (NAC) or a salt thereof, and no less than about 15% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w; wherein the mass ratio of the amino acid, N-(8-[2-hydroxybenzoyl]-amino) caprylic acid (NAC) or its salt, and medium-chain fatty acids or their salts is about 1:(2.5-3.5):(2.5-3.5).

In some embodiments, the medium-chain fatty acid is a fatty acid with a carbon chain length of 6 to 12 carbon atoms, and its structural formula is CH₃(CH₂)ₙCOOH, wherein n is greater than or equal to 4 and less than or equal to 10.

In some embodiments, the medium-chain fatty acid is selected from at least one of caproic acid, enanthic acid, caprylic acid, pelargonic acid, capric acid, undecanoic acid and lauric acid.

In some embodiments, the medium-chain fatty acid salt is selected from at least one of sodium salt, potassium salt and ammonium salt.

In some embodiments, the medium-chain fatty acid salt is sodium caprate.

In some embodiments, the amino acid has nitrogen in its side chain.

In some embodiments, the amino acid comprising nitrogen in the side chain is selected from at least one of lysine, arginine, asparagine, glutamine, tryptophan, ornithine, citrulline, pyrrolysine and histidine.

In some embodiments, the amino acid comprising nitrogen in the side chain is selected from at least one of citrulline and arginine.

In some embodiments, the N-(8-(2-hydroxybenzoyl) amino) caprylic acid (NAC) salt is selected from one or more of N-(8-(2-hydroxybenzoyl) amino) caprylic acid sodium salt, N-(8-(2-hydroxybenzoyl) amino) caprylic acid potassium salt and N-(8-(2-hydroxybenzoyl) amino) caprylic acid ammonium salt.

In some embodiments, the salt of NAC is sodium N-(8-(2-hydroxybenzoyl) amino) caprylate (SNAC).

In some embodiments, the composition comprises:
1-50 part by weight (pbw) of a polypeptide targeting GLP-1 receptor;
15-100 part by weight (pbw) of amino acids;
100-300 part by weight (pbw) of medium-chain fatty acids or their salts;
100-300 part by weight (pbw) of N-(8-[2-hydroxybenzoyl]-amino) caprylic acid (NAC) or its salt.

In some embodiments, the composition comprises:
1-50 part by weight (pbw) of a polypeptide targeting GLP-1 receptor;
20-100 part by weight (pbw) of amino acids;
100-300 part by weight (pbw) of medium-chain fatty acids or their salts;
100-300 part by weight (pbw) of N-(8-[2-hydroxybenzoyl]-amino) caprylic acid (NAC) or its salt.

In some embodiments, the composition comprises:
1-50 part by weight (pbw) of a polypeptide targeting GLP-1 receptor;
30-100 part by weight (pbw) of amino acids;
100-300 part by weight (pbw) of medium-chain fatty acids or their salts;
100-300 part by weight (pbw) of N-(8-[2-hydroxybenzoyl]-amino) caprylic acid (NAC) or its salt.

In some embodiments, the composition comprises:
1-40 pbw of a polypeptide targeting the GLP-1 receptor;
40-70 pbw of amino acids;
120-200 pbw of medium-chain fatty acids or their salts;
120-200 pbw of N-(8-(2-hydroxybenzoyl) amino) caprylic acid (NAC) or its salts.

In some embodiments, the composition is made up of:
1-32 pbw of a polypeptide targeting the GLP-1 receptor;
50-70 pbw of amino acids;
120-180 pbw of medium-chain fatty acids or their salts;
120-180 pbw of N-(8-(2-hydroxybenzoyl) amino) caprylic acid (NAC) or its salts.

In some embodiments, the composition comprises:
5-10 pbw of a polypeptide targeting the GLP-1 receptor;
50 pbw of amino acids;
150 pbw of medium-chain fatty acids or their salts;
150 pbw of N-(8-(2-hydroxybenzoyl) amino) caprylic acid (NAC) or its salts.

In some embodiments, the composition comprises:
1-50 pbw of semaglutide;
15-100 pbw of arginine;
100-300 pbw of sodium caprate;
100-300 pbw of sodium N-(8-(2-hydroxybenzoyl)amino)caprylate (SNAC).

In some embodiments, the composition comprises:
1-50 pbw of semaglutide;
20-100 pbw of arginine;
100-300 pbw of sodium caprate;
100-300 pbw of sodium N-(8-(2-hydroxybenzoyl)amino)caprylate (SNAC).

In some embodiments, the composition comprises:
1-50 pbw of semaglutide;
30-100 pbw of arginine;
100-300 pbw of sodium caprate;
100-300 pbw of sodium N-(8-(2-hydroxybenzoyl)amino)caprylate (SNAC).

In some embodiments, the composition comprises:
1-40 pbw of semaglutide;
40-70 pbw of arginine;
120-200 pbw of sodium caprate;
120-200 pbw of sodium N-(8-(2-hydroxybenzoyl)amino)caprylate (SNAC).

In some embodiments, the composition comprises:
1-32 pbw of semaglutide;
50-70 pbw of arginine;
120-180 pbw of sodium caprate;
120-180 pbw of sodium N-(8-(2-hydroxybenzoyl)amino)caprylate (SNAC).

In some embodiments, the composition further comprises at least one excipient.

In some embodiments, the composition further comprises at least two excipients.

In some embodiments, the composition comprises no more than about 30% w/w excipients.

In some embodiments, the composition comprises no more than about 25% w/w excipients.

In some embodiments, the composition comprises no less than about 10% w/w excipients.

In some embodiments, the composition comprises no less than about 15% w/w excipients.

In some embodiments, the excipients comprise at least one of fillers, disintegrants and lubricants.

In some embodiments, the excipients comprise at least one of fillers, disintegrants, lubricants and binding agents.

In some embodiments, the filler is selected from at least one of starch, powdered sugar, dextrin, lactose, microcrystalline cellulose, pregelatinized starch, mannitol and calcium sulfate.

In some embodiments, the filler is microcrystalline cellulose.

In some embodiments, the lubricant is selected from at least one of magnesium stearate, colloidal silicon dioxide, talcum powder, polyethylene glycol and sodium lauryl sulfate.

In some embodiments, the lubricant is sodium lauryl sulfate.

In some embodiments, the disintegrant is selected from at least one of dry starch, sodium carboxymethyl cellulose, low-substituted sodium carboxymethyl cellulose, cross-linked polyvinylpyrrolidone, cross-linked sodium carboxymethyl cellulose and sodium carboxymethyl starch.

In some embodiments, the disintegrant is sodium carboxymethyl starch.

In some embodiments, the binding agent is selected from at least one of povidone, cross-linked povidone, gum arabic, gelatin, maltodextrin, sodium carboxymethyl cellulose and hydroxypropyl methyl cellulose.

In some embodiments, the binding agent is povidone.

In some embodiments, the composition comprises no less than about 10.0% w/w fillers; such as 11.0% w/w, 12.0% w/w, 15.0% w/w, 16.0% w/w, 17.0% w/w, 18.0% w/w, 18.5% w/w, 19.0% w/w, 19.1% w/w, 19.2% w/w, 19.3% w/w, 19.5% w/w, 19.7% w/w, 19.8% w/w or 20.0% w/w.

In some embodiments, the composition comprises no more than about 5.0% w/w lubricants.

In some embodiments, the composition comprises no more than about 3.0% w/w lubricants.

In some embodiments, the composition comprises no more than about 2.0% w/w lubricants.

In some embodiments, the composition comprises no more than about 1.0% w/w lubricants.

In some embodiments, the composition comprises about 1.0% w/w, 0.9% w/w, 0.96% w/w, 0.95% w/w, 0.89% w/w, 0.8% w/w, 0.7% w/w, 0.6% w/w, 0.5% w/w, 0.1% w/w or 0.01% w/w lubricants.

In some embodiments, the composition comprises no more than about 5.0% w/w disintegrants.

In some embodiments, the composition comprises no more than about 4.0% w/w disintegrants.

In some embodiments, the composition comprises no more than about 3.0% w/w disintegrants.

In some embodiments, the composition comprises about 2.5% w/w, 2.0% w/w, 1.9% w/w, 1.8% w/w, 1.7% w/w, 1.6% w/w, 1.5% w/w, 1.4% w/w, 1.2% w/w, 1.0% w/w, 0.9% w/w, 0.8% w/w, 0.5% w/w or 0.1% w/w disintegrants.

In some embodiments, the composition comprises no more than about 5.0% w/w binding agents.

In some embodiments, the composition comprises about 4.0% w/w, 3.5% w/w, 3.3% w/w, 3.1% w/w, 3.0% w/w, 2.9% w/w, 2.5% w/w, 2.3% w/w, 2.0% w/w, 1.9% w/w, 1.8% w/w, 1.7% w/w, 1.6% w/w, 1.5% w/w, 1.3% w/w, 1.2% w/w, 1.0% w/w, 0.1% w/w or 0.01% w/w binding agents.

In some embodiments, the excipients comprise microcrystalline cellulose, sodium lauryl sulfate and sodium carboxymethyl starch.

In some embodiments, the excipients comprise microcrystalline cellulose, sodium lauryl sulfate, sodium carboxymethyl starch and povidone.

In some embodiments, the excipients comprise about 10.0-25.0% w/w of microcrystalline cellulose.

In some embodiments, the excipients comprise about 0.01-3.0% w/w of sodium lauryl sulfate.

In some embodiments, the excipients comprise about 0.1-5.0% w/w of sodium carboxymethyl starch.

In some embodiments, the excipients comprise about 0.01-5.0% w/w of povidone.

In some embodiments, the excipients comprise about 10.0-25.0% w/w microcrystalline cellulose, about 0.01-3.0% w/w sodium lauryl sulfate and about 0.1%-5.0% w/w sodium carboxymethyl starch.

In some embodiments, the excipients comprise about 10.0-25.0% w/w microcrystalline cellulose, about 0.01-3.0% w/w sodium lauryl sulfate, about 0.1-5.0% w/w sodium carboxymethyl starch and about 0.01-5.0% w/w povidone.

In some embodiments, the composition comprises:
1-50 pbw of a polypeptide targeting GLP-1 receptor;
15-100 pbw of amino acids;
100-300 pbw of medium-chain fatty acids or their salts;
100-300 pbw of N-(8-[2-hydroxybenzoyl]-amino) caprylic acid (NAC) or its salt;
40-180 pbw of excipients.

In some embodiments, the composition comprises:
1-50 pbw of a polypeptide targeting GLP-1 receptor;
20-100 pbw of amino acids;
100-300 pbw of medium-chain fatty acids or their salts;
100-300 pbw of N-(8-[2-hydroxybenzoyl]-amino) caprylic acid (NAC) or its salt;
40-180 pbw of excipients.

In some embodiments, the composition comprises:
1-50 pbw of a polypeptide targeting GLP-1 receptor;
30-100 pbw of amino acids;
100-300 pbw of medium-chain fatty acids or their salts;
100-300 pbw of N-(8-[2-hydroxybenzoyl]-amino) caprylic acid (NAC) or its salt;
40-180 pbw of excipients.

In some embodiments, the composition comprises:
1-40 pbw of a polypeptide targeting the GLP-1 receptor;
40-70 pbw of amino acids;
120-200 pbw of medium-chain fatty acids or their salts;
120-200 pbw of N-(8-(2-hydroxybenzoyl) amino) caprylic acid (NAC) or its salts;
50-160 pbw of excipients.

In some embodiments, the composition is made up of:
1-32 pbw of a polypeptide targeting the GLP-1 receptor;
50-70 pbw of amino acids;
120-180 pbw of medium-chain fatty acids or their salts;
120-180 pbw of N-(8-(2-hydroxybenzoyl) amino) caprylic acid (NAC) or its salts;
70-140 pbw of excipients.

In some embodiments, the composition comprises:
5-10 pbw of a polypeptide targeting the GLP-1 receptor;
50 pbw of amino acids;
150 pbw of medium-chain fatty acids or their salts;
150 pbw of N-(8-(2-hydroxybenzoyl) amino) caprylic acid (NAC) or its salts;
105 pbw of excipients.

In some embodiments, the composition comprises:
1-50 pbw of semaglutide;
15-100 pbw of arginine;
100-300 pbw of sodium caprate;
100-300 pbw of sodium N-(8-(2-hydroxybenzoyl)amino)caprylate (SNAC);
40-180 pbw of excipients.

In some embodiments, the composition comprises:
1-50 pbw of semaglutide;
20-100 pbw of arginine;
100-300 pbw of sodium caprate;
100-300 pbw of sodium N-(8-(2-hydroxybenzoyl)amino)caprylate (SNAC);
40-180 pbw of excipients.

In some embodiments, the composition comprises:
1-50 pbw of semaglutide;
30-100 pbw of arginine;
100-300 pbw of sodium caprate;
100-300 pbw of sodium N-(8-(2-hydroxybenzoyl)amino)caprylate (SNAC);
40-180 pbw of excipients.

In some embodiments, the composition comprises:
1-40 pbw of semaglutide;
40-70 pbw of arginine;
120-200 pbw of sodium caprate;
120-200 pbw of sodium N-(8-(2-hydroxybenzoyl)amino)caprylate (SNAC);
50-160 pbw of excipients.

In some embodiments, the composition comprises:
1-32 pbw of semaglutide;
50-70 pbw of arginine;
120-180 pbw of sodium caprate;
120-180 pbw of sodium N-(8-(2-hydroxybenzoyl)amino)caprylate (SNAC);
70-140 pbw of excipients.

In some embodiments, the composition comprises:
5 pbw of semaglutide;
50 pbw of arginine;
150 pbw of sodium caprate;
150 pbw of sodium N-(8-(2-hydroxybenzoyl)amino)caprylate (SNAC);
105 pbw of excipients.

In some embodiments, the composition comprises:
4 pbw of semaglutide;
50 pbw of arginine;
150 pbw of sodium caprate;
150 pbw of sodium N-(8-(2-hydroxybenzoyl)amino)caprylate (SNAC);
124 pbw of excipients.

In some embodiments, the composition comprises:
8 pbw of semaglutide;
50 pbw of arginine;
150 pbw of sodium caprate;
150 pbw of sodium N-(8-(2-hydroxybenzoyl)amino)caprylate (SNAC);
120 pbw of excipients.

In some embodiments, the composition comprises:
16 pbw of semaglutide;
50 pbw of arginine;
150 pbw of sodium caprate;
150 pbw of sodium N-(8-(2-hydroxybenzoyl)amino)caprylate (SNAC);
110 pbw of excipients.

In some embodiments, the composition comprises:
1-50 pbw of a polypeptide targeting GLP-1 receptor;
20-100 pbw of amino acids;
100-300 pbw of medium-chain fatty acids or their salts;
100-300 pbw of N-(8-[2-hydroxybenzoyl]-amino) caprylic acid (NAC) or its salt;
50-150 pbw of fillers;
1-15 pbw of lubricants; and
1-30 pbw of disintegrants.

In some embodiments, the composition comprises:
1-50 pbw of a polypeptide targeting GLP-1 receptor;
30-100 pbw of amino acids;
100-300 pbw of medium-chain fatty acids or their salts;
100-300 pbw of N-(8-[2-hydroxybenzoyl]-amino) caprylic acid (NAC) or its salt;
50-150 pbw of fillers;
1-15 pbw of lubricants; and
1-30 pbw of disintegrants.

In some embodiments, the composition comprises:
1-40 pbw of a polypeptide targeting the GLP-1 receptor;
40-70 pbw of amino acids;
120-200 pbw of medium-chain fatty acids or their salts;
120-200 pbw of N-(8-(2-hydroxybenzoyl) amino) caprylic acid (NAC) or its salts;
40-140 pbw of fillers;
1-15 pbw of lubricants; and
1-30 pbw of disintegrants.

In some embodiments, the composition is made up of:
1-32 pbw of a polypeptide targeting the GLP-1 receptor;
50-70 pbw of amino acids;
120-180 pbw of medium-chain fatty acids or their salts;
120-180 pbw of N-(8-(2-hydroxybenzoyl) amino) caprylic acid (NAC) or its salts;
80-130 pbw of fillers;
1-15 pbw of lubricants; and
1-30 pbw of disintegrants.

In some embodiments, the composition comprises:
5-10 pbw of a polypeptide targeting the GLP-1 receptor;
50 pbw of amino acids;
150 pbw of medium-chain fatty acids or their salts;
150 pbw of N-(8-(2-hydroxybenzoyl) amino) caprylic acid (NAC) or its salts;
80-110 pbw of fillers;
1-10 pbw of lubricants; and
1-15 pbw of disintegrants.

In some embodiments, the composition comprises:
1-50 pbw of semaglutide;
20-100 pbw of arginine;
100-300 pbw of sodium caprate;
100-300 pbw of sodium N-(8-(2-hydroxybenzoyl)amino)caprylate (SNAC);
50-150 pbw of fillers;
1-15 pbw of lubricants; and
1-30 pbw of disintegrants.

In some embodiments, the composition comprises:
1-50 pbw of semaglutide;
30-100 pbw of arginine;
100-300 pbw of sodium caprate;
100-300 pbw of sodium N-(8-(2-hydroxybenzoyl)amino)caprylate (SNAC);
50-150 pbw of fillers;
1-15 pbw of lubricants; and
1-30 pbw of disintegrants.

In some embodiments, the composition comprises:
1-40 pbw of semaglutide;
40-70 pbw of arginine;
120-200 pbw of sodium caprate;
120-200 pbw of sodium N-(8-(2-hydroxybenzoyl)amino)caprylate (SNAC);
70-140 pbw of fillers;
1-15 pbw of lubricants; and
1-30 pbw of disintegrants.

In some embodiments, the composition comprises:
5-10 pbw of semaglutide;
50 pbw of arginine;
150 pbw of sodium caprate;
150 pbw of sodium N-(8-(2-hydroxybenzoyl)amino)caprylate (SNAC);
80-110 pbw of fillers;
1-10 pbw of lubricants; and
1-15 pbw of disintegrants.

In some embodiments, the composition comprises:
1-50 pbw of a polypeptide targeting GLP-1 receptor;
30-100 pbw of amino acids;
100-300 pbw of medium-chain fatty acids or their salts;
100-300 pbw of N-(8-[2-hydroxybenzoyl]-amino) caprylic acid (NAC) or its salt;
50-150 pbw of fillers;
1-15 pbw of lubricants;
1-30 pbw of disintegrants; and
1-30 pbw of binding agents.

In some embodiments, the composition comprises:
1-40 pbw of a polypeptide targeting the GLP-1 receptor;
40-70 pbw of amino acids;
120-200 pbw of medium-chain fatty acids or their salts;
120-200 pbw of N-(8-(2-hydroxybenzoyl) amino) caprylic acid (NAC) or its salts;
70-140 pbw of fillers;
1-15 pbw of lubricants;
1-30 pbw of disintegrants; and
1-30 pbw of binding agents.

In some embodiments, the composition is made up of:
1-32 pbw of a polypeptide targeting the GLP-1 receptor;
50-70 pbw of amino acids;
120-180 pbw of medium-chain fatty acids or their salts;
120-180 pbw of N-(8-(2-hydroxybenzoyl) amino) caprylic acid (NAC) or its salts;
80-130 pbw of fillers;
1-10 pbw of lubricants;
1-15 pbw of disintegrants; and
1-15 pbw of binding agents.

In some embodiments, the composition comprises:
5-10 pbw of a polypeptide targeting the GLP-1 receptor;
50 pbw of amino acids;
150 pbw of medium-chain fatty acids or their salts;
150 pbw of N-(8-(2-hydroxybenzoyl) amino) caprylic acid (NAC) or its salts;
80-110 pbw of fillers;
1-10 pbw of lubricants;
1-15 pbw of disintegrants; and
1-15 pbw of binding agents.

In some embodiments, the composition comprises: about 4.0 mg semaglutide, about 50.0 mg arginine, about 150.0 mg sodium N-(8-[2-hydroxybenzoyl]-amino)caprylate, about 150.0 mg sodium caprate, and about 123.0 mg excipients.

In some embodiments, the composition comprises: about 8.0 mg semaglutide, about 50.0 mg arginine, about 150.0 mg sodium N-(8-[2-hydroxybenzoyl]-amino)caprylate, about 150.0 mg sodium caprate, and about 120.0 mg excipients.

In some embodiments, the composition comprises: about 16.0 mg semaglutide, about 50.0 mg arginine, about 150.0 mg sodium N-(8-[2-hydroxybenzoyl]-amino)caprylate, about 150.0 mg sodium caprate, and about 110.0 mg excipients.

In some embodiments, the composition is a solid preparation.

In some embodiments, the composition is tablets, capsules, granules, powders, granules, pellets or pills.

In some embodiments, the composition is tablets.

In some embodiments, the specification of the tablet is about 1-50 mg.

In some embodiments, the specification of the tablet is about 1-40 mg.

In some embodiments, the specification of the tablet is about 1-30 mg.

In some embodiments, the specification of the tablet is about 5-10 mg.

In some embodiments, the specification of the tablet is about 1 mg, 2 mg, 3 mg, 4 mg, 5 mg, 6 mg, 6.5 mg, 6.8 mg, 7 mg, 7.1 mg, 7.4 mg, 7.5 mg, 7.9 mg, 8 mg, 8.6 mg, 9 mg, 10 mg, 14 mg, 15 mg, 16 mg, 17 mg, 18 mg, 19 mg, 20 mg, 21 mg, 22 mg, 23 mg, 24 mg, 25 mg, 26 mg, 28 mg, 30 mg, 31 mg, 32 mg, 33 mg, 34 mg, 35 mg or 40 mg.

Another aspect of the present disclosure provides an oral polypeptide composition comprising a polypeptide targeting GLP-1 receptor use for (i) treating a disease; and/or (ii) preparing a drug for treating a disease; wherein, the disease is selected from at least one of type 1 diabetes, type 2 diabetes, obesity, cardiovascular disease, non-alcoholic fatty liver disease, chronic kidney disease, Alzheimer's disease, cancer, polycystic ovary syndrome, metabolic syndrome, bone metabolism and osteoporosis, knee osteoarthritis, sleep apnea syndrome, osteoarthritis and metabolic muscle disease.

Another aspect of the present disclosure provides a method for preparing an oral polypeptide tablet, comprising the steps of: directly tableting the oral polypeptide composition of the present disclosure to obtain a polypeptide tablet.

Another aspect of the present disclosure provides a composition for pharmaceutical preparation, wherein the composition comprising a mixed granules A and granules B, wherein the granules A comprises a polypeptide targeting GLP-1 receptor; the granules B comprises one or more amino acids, N-(8-(2-hydroxybenzoyl)amino)caprylic acid (NAC) or a salt thereof, and one or more medium-chain fatty acids or salts thereof.

In some embodiments, the granules Aare granulated from a composition that comprises a polypeptide targeting GLP-1 receptor; and/or the granules B are granulated from a composition comprises one or more amino acids, N-(8-(2-hydroxybenzoyl)amino)caprylic acid (NAC) or a salt thereof, and one or more medium-chain fatty acids or salts thereof.

In some embodiments, the granules Aare granulated from a composition that comprises a polypeptide targeting GLP-1 receptor and at least one excipient.

In some embodiments, the granules A comprise no less than about 10% w/w the polypeptide targeting GLP-1 receptor, such as 10% w/w, 20% w/w, 25% w/w, 30% w/w, 40% w/w, 42% w/w or 45% w/w.

In some embodiments, the granules A comprise no less than about 50% w/w excipients.

In some embodiments, the granules A comprise a polypeptide targeting GLP-1 receptor, fillers and binding agents.

In some embodiments, the granules B comprise one or more amino acids, N-(8-(2-hydroxybenzoyl)amino)caprylic acid (NAC) or a salt thereof, one or more medium-chain fatty acids or salts thereof, and at least one excipient.

In some embodiments, the granules B comprise one or more amino acids, N-(8-(2-hydroxybenzoyl)amino)caprylic acid (NAC) or a salt thereof, one or more medium-chain fatty acids or salts thereof, fillers, lubricants and disintegrants.

In some embodiments, the mass percentage of the amino acid, SNAC, and sodium caprate in granules B is at least about 60%.

In some embodiments, the mass percentage of the amino acid, SNAC, and sodium caprate in granules B is at least about 70%.

In some embodiments, the granules B comprise no less than about 10% w/w excipients.

In some embodiments, the granules B comprise no less than about 20% w/w excipients.

In some embodiments, the mass percentage of the granules A in the composition is no more than about 20% w/w.

In some embodiments, the mass percentage of the granules A in the composition is no more than about 10% w/w.

In some embodiments, the mass percentage of the granules B in the composition is at least about 80% w/w.

In some embodiments, the mass percentage of the granules B in the composition is at least about 90% w/w.

In some embodiments, the composition for pharmaceutical preparation comprises no more than about 55% w/w N-(8-[2-hydroxybenzoyl]-amino) caprylic acids (NAC) or salts thereof.

In some embodiments, the composition for pharmaceutical preparation comprises no more than about 50% w/w N-(8-[2-hydroxybenzoyl]-amino) caprylic acids (NAC) or salts thereof.

In some embodiments, the composition for pharmaceutical preparation comprises no more than about 45% w/w N-(8-[2-hydroxybenzoyl]-amino) caprylic acid (NAC) or salts thereof.

In some embodiments, the composition for pharmaceutical preparation comprises no more than about 40% w/w N-(8-[2-hydroxybenzoyl]-amino) caprylic acid (NAC) or salts thereof.

In some embodiments, the composition for pharmaceutical preparation comprises no more than about 35% w/w N-(8-[2-hydroxybenzoyl]-amino) caprylic acid (NAC) or salts thereof.

In some embodiments, the composition for pharmaceutical preparation comprises about 49% w/w, 48% w/w, 47% w/w, 46% w/w, 45% w/w, 44% w/w, 43% w/w, 42% w/w, 41% w/w, 40% w/w, 39% w/w, 38% w/w, 37% w/w, 36% w/w or 35% w/w N-(8-[2-hydroxybenzoyl]-amino)caprylic acid (NAC) or salts thereof.

In some embodiments, the composition for pharmaceutical preparation comprises no less than about 20% w/w N-(8-[2-hydroxybenzoyl]-amino) caprylic acid (NAC) or salts thereof.

In some embodiments, the composition for pharmaceutical preparation comprises no less than about 25% w/w N-(8-[2-hydroxybenzoyl]-amino) caprylic acid (NAC) or salts thereof.

In some embodiments, the composition for pharmaceutical preparation comprises no less than about 30% w/w N-(8-[2-hydroxybenzoyl]-amino) caprylic acid (NAC) or salts thereof.

In some embodiments, the composition for pharmaceutical preparation comprises about 21% w/w, 22% w/w, 23% w/w, 24% w/w, 25% w/w, 26% w/w, 28% w/w, 29% w/w or 30% w/w N-(8-[2-hydroxybenzoyl]-amino) caprylic acid (NAC) or salts thereof.

In some embodiments, the composition for pharmaceutical preparation comprises about 20-50% w/w N-(8-[2-hydroxybenzoyl]-amino) caprylic acid (NAC) or salts thereof.

In some embodiments, the composition for pharmaceutical preparation comprises about 20-45% w/w N-(8-[2-hydroxybenzoyl]-amino) caprylic acid (NAC) or salts thereof.

In some embodiments, the composition for pharmaceutical preparation comprises about 20-40% w/w N-(8-[2-hydroxybenzoyl]-amino) caprylic acid (NAC) or salts thereof.

In some embodiments, the composition for pharmaceutical preparation comprises about 25-50% w/w N-(8-[2-hydroxybenzoyl]-amino) caprylic acid (NAC) or salts thereof.

In some embodiments, the composition for pharmaceutical preparation comprises about 25-45% w/w N-(8-[2-hydroxybenzoyl]-amino) caprylic acid (NAC) or salts thereof.

In some embodiments, the composition for pharmaceutical preparation comprises about 25-40% w/w N-(8-[2-hydroxybenzoyl]-amino) caprylic acid (NAC) or salts thereof.

In some embodiments, the composition for pharmaceutical preparation comprises about 30-50% w/w N-(8-[2-hydroxybenzoyl]-amino) caprylic acid (NAC) or salts thereof.

In some embodiments, the composition for pharmaceutical preparation comprises about 30-45% w/w N-(8-[2-hydroxybenzoyl]-amino) caprylic acid (NAC) or salts thereof.

In some embodiments, the composition for pharmaceutical preparation comprises about 30-40% w/w N-(8-[2-hydroxybenzoyl]-amino) caprylic acid (NAC) or salts thereof.

In some embodiments, the composition for pharmaceutical preparation comprises about 30-35% w/w N-(8-[2-hydroxybenzoyl]-amino) caprylic acid (NAC) or salts thereof.

In some embodiments, the composition for pharmaceutical preparation comprises no more than about 20% w/w amino acid.

In some embodiments, the composition for pharmaceutical preparation comprises no more than about 18% w/w amino acid.

In some embodiments, the composition for pharmaceutical preparation comprises no more than about 15% w/w amino acid.

In some embodiments, the composition for pharmaceutical preparation comprises no more than about 15% w/w amino acid.

In some embodiments, the composition for pharmaceutical preparation comprises no more than about 19% w/w, 18% w/w, 17% w/w, 16% w/w, 15% w/w, 14% w/w, 13% w/w or 12% w/w amino acid.

In some embodiments, the composition for pharmaceutical preparation comprises no less than about 7% w/w amino acid.

In some embodiments, the composition for pharmaceutical preparation comprises no less than about 8% w/w amino acid.

In some embodiments, the composition for pharmaceutical preparation comprises no less than about 9% w/w amino acid.

In some embodiments, the composition for pharmaceutical preparation comprises no less than about 10% w/w amino acid.

In some embodiments, the composition for pharmaceutical preparation comprises no less than about 7.5% w/w, 7.8% w/w, 8.3% w/w, 8.5% w/w, 8.8% w/w, 9% w/w, 9.2% w/w, 9.5% w/w or 10% w/w amino acid.

In some embodiments, the composition for pharmaceutical preparation comprises about 7-20% w/w amino acid.

In some embodiments, the composition for pharmaceutical preparation comprises about 7-18% w/w amino acid.

In some embodiments, the composition for pharmaceutical preparation comprises about 7-15% w/w amino acid.

In some embodiments, the composition for pharmaceutical preparation comprises about 8-20% w/w amino acid.

In some embodiments, the composition for pharmaceutical preparation comprises about 8-18% w/w amino acid.

In some embodiments, the composition for pharmaceutical preparation comprises about 8-15% w/w amino acid.

In some embodiments, the composition for pharmaceutical preparation comprises about 9-20% w/w amino acid.

In some embodiments, the composition for pharmaceutical preparation comprises about 10-20% w/w amino acid.

In some embodiments, the composition for pharmaceutical preparation comprises about 9-15% w/w amino acid.

In some embodiments, the composition for pharmaceutical preparation comprises about 10-15% w/w amino acid.

In some embodiments, the composition for pharmaceutical preparation comprises no more than about 40% w/w medium-chain fatty acids or salts thereof.

In some embodiments, the composition for pharmaceutical preparation comprises no more than about 35% w/w medium-chain fatty acids or salts thereof.

In some embodiments, the composition for pharmaceutical preparation comprises no more than about 39% w/w, 38% w/w, 37% w/w, 36% w/w or 35% w/w medium-chain fatty acids or salts thereof.

In some embodiments, the composition for pharmaceutical preparation comprises no less than 10% w/w medium-chain fatty acids or salts thereof.

In some embodiments, the composition for pharmaceutical preparation comprises no less than 15% w/w medium-chain fatty acids or salts thereof.

In some embodiments, the composition for pharmaceutical preparation comprises no less than 20% w/w medium-chain fatty acids or salts thereof.

In some embodiments, the composition for pharmaceutical preparation comprises no less than 30% w/w medium-chain fatty acids or salts thereof.

In some embodiments, the composition for pharmaceutical preparation comprises no less than 10% w/w, 12% w/w, 14% w/w, 15% w/w, 16% w/w, 18% w/w, 21% w/w, 22% w/w, 23% w/w, 24% w/w, 25% w/w, 26% w/w, 28% w/w, 29% w/w or 30% w/w medium-chain fatty acids or salts thereof.

In some embodiments, the composition for pharmaceutical preparation comprises about 10-40% w/w medium-chain fatty acids or salts thereof.

In some embodiments, the composition for pharmaceutical preparation comprises about 15-40% w/w medium-chain fatty acids or salts thereof.

In some embodiments, the composition for pharmaceutical preparation comprises about 15-35% w/w medium-chain fatty acids or salts thereof.

In some embodiments, the composition for pharmaceutical preparation comprises about 20-40% w/w medium-chain fatty acids or salts thereof.

In some embodiments, the composition for pharmaceutical preparation comprises about 25-35% w/w medium-chain fatty acids or salts thereof.

In some embodiments, the composition for pharmaceutical preparation comprises about 25-40% w/w medium-chain fatty acids or salts thereof.

In some embodiments, the composition for pharmaceutical preparation comprises about 30-40% w/w medium-chain fatty acids or salts thereof.

In some embodiments, the composition for pharmaceutical preparation comprises about 30-35% w/w medium-chain fatty acids or salts thereof.

In some embodiments, the composition for pharmaceutical preparation comprises about no more than about 15% w/w polypeptide targeting the GLP-1 receptor.

In some embodiments, the composition for pharmaceutical preparation comprises about no more than about 10% w/w polypeptide targeting the GLP-1 receptor.

In some embodiments, the composition for pharmaceutical preparation comprises about 0.1-15% w/w polypeptide targeting the GLP-1 receptor.

In some embodiments, the composition for pharmaceutical preparation comprises about 0.5-10% w/w polypeptide targeting the GLP-1 receptor.

In some embodiments, the composition for pharmaceutical preparation comprises about 0.8-10% w/w polypeptide targeting the GLP-1 receptor.

In some embodiments, the composition for pharmaceutical preparation comprises about 1.0-7.0% w/w polypeptide targeting the GLP-1 receptor.

In some embodiments, the composition for pharmaceutical preparation comprises about 0.2% w/w, 0.8% w/w, 0.9% w/w, 1.0% w/w, 1.5% w/w, 1.6% w/w, 1.7% w/w, 1.8% w/w, 3.3% w/w, 3.4% w/w, 6.9% w/w, 7.0% w/w, 8.0% w/w, 9.0% w/w, 10.0% w/w polypeptide targeting the GLP-1 receptor.

In some embodiments, the total content of the amino acids, N-(8-[2-hydroxybenzoyl]-amino) caprylic acid (NAC) or a salt thereof and medium-chain fatty acids or salts thereof in the composition for pharmaceutical preparation is no more than about 90% w/w.

In some embodiments, the total content of the amino acids, N-(8-[2-hydroxybenzoyl]-amino) caprylic acid (NAC) or a salt thereof and medium-chain fatty acids or salts thereof in the composition for pharmaceutical preparation is no more than about 85% w/w.

In some embodiments, the total content of the amino acids, N-(8-[2-hydroxybenzoyl]-amino) caprylic acid (NAC) or a salt thereof and medium-chain fatty acids or salts thereof in the composition for pharmaceutical preparation is no more than about 80% w/w.

In some embodiments, the total content of the amino acids, N-(8-[2-hydroxybenzoyl]-amino) caprylic acid (NAC) or a salt thereof and medium-chain fatty acids or salts thereof in the composition for pharmaceutical preparation is no less than about 50% w/w.

In some embodiments, the total content of the amino acids, N-(8-[2-hydroxybenzoyl]-amino) caprylic acid (NAC) or a salt thereof and medium-chain fatty acids or salts thereof in the composition for pharmaceutical preparation is no less than about 60% w/w.

In some embodiments, the total content of the amino acids, N-(8-[2-hydroxybenzoyl]-amino) caprylic acid (NAC) or a salt thereof and medium-chain fatty acids or salts thereof in the composition for pharmaceutical preparation is no less than about 70% w/w.

In some embodiments, the total content of the amino acids, N-(8-[2-hydroxybenzoyl]-amino) caprylic acid (NAC) or a salt thereof and medium-chain fatty acids or salts thereof in the composition for pharmaceutical preparation is about 50-80% w/w.

In some embodiments, the total content of the amino acids, N-(8-[2-hydroxybenzoyl]-amino) caprylic acid (NAC) or a salt thereof and medium-chain fatty acids or salts thereof in the composition for pharmaceutical preparation is about 60-80% w/w.

In some embodiments, the total content of the amino acids, N-(8-[2-hydroxybenzoyl]-amino) caprylic acid (NAC) or a salt thereof and medium-chain fatty acids or salts thereof in the composition for pharmaceutical preparation is about 70-80% w/w.

In some embodiments, the total content of the amino acids, N-(8-[2-hydroxybenzoyl]-amino) caprylic acid (NAC) or a salt thereof and medium-chain fatty acids or salts thereof in the composition for pharmaceutical preparation is about 60% w/w, 62% w/w, 63% w/w, 64% w/w, 65% w/w, 67% w/w, 68% w/w, 70% w/w, 71% w/w, 72% w/w, 73% w/w, 74% w/w, 75% w/w, 75.5% w/w, 75.8% w/w, 75.9% w/w, 77% w/w, 79% w/w or 80% w/w.

In some embodiments, in the composition for pharmaceutical preparation, the mass percentage of the amino acids does not exceed that of N-(8-[2-hydroxybenzoyl]-amino) caprylic acid (NAC) or its salt.

In some embodiments, in the composition for pharmaceutical preparation, the mass percentage of the amino acids does not exceed that of medium-chain fatty acids or their salts.

In some embodiments, in the composition for pharmaceutical preparation, the mass percentage of the medium-chain fatty acids or their salts does not exceed that of N-(8-[2-hydroxybenzoyl]-amino) caprylic acid (NAC) or its salt.

In some embodiments, in the composition for pharmaceutical preparation, the mass ratio of the amino acid, N-(8-[2-hydroxybenzoyl]-amino) caprylic acid (NAC) or its salt and the medium-chain fatty acids or their salts is about (1-3):3:(1.5-6).

In some embodiments, in the composition for pharmaceutical preparation, the mass ratio of the N-(8-[2-hydroxybenzoyl]-amino) caprylic acid (NAC) or its salt to the medium-chain fatty acids or their salts is about 1:(0.5-2), such as 1:1, 2:1, 1.5:1, 1:1.5.

In some embodiments, in the composition for pharmaceutical preparation, the mass ratio of the amino acid to the N-(8-[2-hydroxybenzoyl]-amino) caprylic acid (NAC) or its salt is about (1-2):3, such as 1:3, 2:3, 1.5:3.

In some embodiments, in the composition for pharmaceutical preparation, the mass ratio of the amino acid, N-(8-[2-hydroxybenzoyl]-amino) caprylic acid (NAC) or its salt, and medium-chain fatty acids or their salts is about 1:(2.5-3.5):(2.5-3.5).

In some embodiments, in the composition for pharmaceutical preparation, the mass ratio of the amino acid, N-(8-[2-hydroxybenzoyl]-amino) caprylic acid (NAC) or its salt, and medium-chain fatty acids or their salts is about 1:(2.6-3.4):(2.6-3.4).

In some embodiments, in the composition for pharmaceutical preparation, the mass ratio of the amino acid, N-(8-[2-hydroxybenzoyl]-amino) caprylic acid (NAC) or its salt, and medium-chain fatty acids or their salts is about 1:(2.7-3.3):(2.7-3.3).

In some embodiments, in the composition for pharmaceutical preparation, the mass ratio of the amino acid, N-(8-[2-hydroxybenzoyl]-amino) caprylic acid (NAC) or its salt, and medium-chain fatty acids or their salts is about 1:(2.8-3.2):(2.8-3.2).

In some embodiments, in the composition for pharmaceutical preparation, the mass ratio of the amino acid, N-(8-[2-hydroxybenzoyl]-amino) caprylic acid (NAC) or its salt, and medium-chain fatty acids or their salts is about 1:(2.9-3.1):(2.9-3.1).

In some embodiments, in the composition for pharmaceutical preparation, the mass ratio of the amino acid, N-(8-[2-hydroxybenzoyl]-amino) caprylic acid (NAC) or its salt, and medium-chain fatty acids or their salt is about 1:3:3.

In some embodiments, in the composition for pharmaceutical preparation, the mass ratio of N-(8-[2-hydroxybenzoyl]-amino) caprylic acid (NAC) or its salt and the medium-chain fatty acids or their salts is about 1:1.

In some embodiments, in the composition for pharmaceutical preparation, the polypeptide targeting GLP-1 receptor is a GLP-1 receptor agonist.

In some embodiments, in the composition for pharmaceutical preparation, the polypeptide targeting GLP-1 receptor is semaglutide.

In some embodiments, the composition for pharmaceutical preparation comprises: semaglutide, no more than about 20% w/w amino acids, no more than about 55% w/w N-(8-[2-hydroxybenzoyl]-amino) caprylic acid (NAC) or its salt, and medium-chain fatty acids or their salts.

In some embodiments, the composition for pharmaceutical preparation comprises: semaglutide, no more than about 20% w/w amino acids, no more than about 55% w/w N-(8-[2-hydroxybenzoyl]-amino) caprylic acid (NAC) or its salt, and no less than about 10% w/w medium-chain fatty acids or their salts.

In some embodiments, the composition for pharmaceutical preparation comprises: semaglutide, no more than about 20% w/w amino acids, no more than about 55% w/w N-(8-[2-hydroxybenzoyl]-amino) caprylic acid (NAC) or its salt, and no less than about 15% w/w medium-chain fatty acids or their salts.

In some embodiments, the composition for pharmaceutical preparation comprises: semaglutide, no more than about 20% w/w amino acids, no more than about 50% w/w N-(8-[2-hydroxybenzoyl]-amino) caprylic acid (NAC) or its salt, and no less than about 10% w/w medium-chain fatty acids or their salts.

In some embodiments, the composition for pharmaceutical preparation comprises: semaglutide, about 10-20% w/w amino acids, about 20-55% w/w N-(8-[2-hydroxybenzoyl]-amino) caprylic acid (NAC) or its salt, and no less than about 10% w/w medium-chain fatty acids or their salts.

In some embodiments, the composition for pharmaceutical preparation comprises: semaglutide, about 10-20% w/w amino acids, about 20-55% w/w N-(8-[2-hydroxybenzoyl]-amino) caprylic acid (NAC) or its salt, and no less than about 15% w/w medium-chain fatty acids or their salts.

In some embodiments, the composition for pharmaceutical preparation comprises: semaglutide, one or more amino acids, N-(8-[2-hydroxybenzoyl]-amino)caprylic acid (NAC) or a salt thereof, and one or more medium-chain fatty acids or salts thereof; wherein the total content of the amino acids, N-(8-[2-hydroxybenzoyl]-amino) caprylic acid (NAC) or a salt thereof and medium-chain fatty acids or salts thereof in the composition is no more than about 90% w/w.

In some embodiments, the composition for pharmaceutical preparation comprises: semaglutide, one or more amino acids, N-(8-[2-hydroxybenzoyl]-amino)caprylic acid (NAC) or a salt thereof, and one or more medium-chain fatty acids or salts thereof; wherein the total content of the amino acids, N-(8-[2-hydroxybenzoyl]-amino) caprylic acid (NAC) or a salt thereof and medium-chain fatty acids or salts thereof in the composition is about 50-90% w/w.

In some embodiments, the composition for pharmaceutical preparation comprises: semaglutide, about 10-20% w/w amino acids, N-(8-[2-hydroxybenzoyl]-amino)caprylic acid (NAC) or a salt thereof, and one or more medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is no more than about 90% w/w.

In some embodiments, the composition for pharmaceutical preparation comprises: semaglutide, about 10-20% w/w amino acids, N-(8-[2-hydroxybenzoyl]-amino)caprylic acid (NAC) or a salt thereof, and one or more medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 50-90% w/w.

In some embodiments, the composition for pharmaceutical preparation comprises: semaglutide, about 10-20% w/w amino acids, N-(8-[2-hydroxybenzoyl]-amino)caprylic acid (NAC) or a salt thereof, and one or more medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is no more than about 80% w/w.

In some embodiments, the composition for pharmaceutical preparation comprises: semaglutide, about 10-20% w/w amino acids, about 20-55% N-(8-[2-hydroxybenzoyl]-amino)caprylic acid (NAC) or a salt thereof, and one or more medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is no more than about 80% w/w.

In some embodiments, the composition for pharmaceutical preparation comprises: semaglutide, about 10-20% w/w amino acids, about 20-55% N-(8-[2-hydroxybenzoyl]-amino)caprylic acid (NAC) or a salt thereof, and no less than about 10% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is no more than about 90% w/w.

In some embodiments, the composition for pharmaceutical preparation comprises: semaglutide, about 10-20% w/w amino acids, about 20-50% N-(8-[2-hydroxybenzoyl]-amino)caprylic acid (NAC) or a salt thereof, and no less than about 15% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is no more than about 90% w/w.

In some embodiments, the composition for pharmaceutical preparation comprises: semaglutide, about 10-20% w/w amino acids, about 20-55% N-(8-[2-hydroxybenzoyl]-amino)caprylic acid (NAC) or a salt thereof, and no less than about 10% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is no more than about 80% w/w.

In some embodiments, the composition for pharmaceutical preparation comprises: semaglutide, about 10-20% w/w amino acids, about 20-50% N-(8-[2-hydroxybenzoyl]-amino)caprylic acid (NAC) or a salt thereof, and no less than about 15% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is no more than about 80% w/w.

In some embodiments, the composition for pharmaceutical preparation comprises: semaglutide, about 10-20% w/w amino acids, about 20-55% N-(8-[2-hydroxybenzoyl]-amino)caprylic acid (NAC) or a salt thereof, and no less than about 10% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w.

In some embodiments, the composition for pharmaceutical preparation comprises: semaglutide, about 10-20% w/w amino acids, about 20-50% N-(8-[2-hydroxybenzoyl]-amino)caprylic acid (NAC) or a salt thereof, and no less than about 15% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w.

In some embodiments, the composition for pharmaceutical preparation comprises: semaglutide, about 10-20% w/w amino acids, about 20-55% N-(8-[2-hydroxybenzoyl]-amino)caprylic acid (NAC) or a salt thereof, and about 10-40% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w.

In some embodiments, the composition for pharmaceutical preparation comprises: semaglutide, about 10-20% w/w amino acids, about 20-50% N-(8-[2-hydroxybenzoyl]-amino)caprylic acid (NAC) or a salt thereof, and about 15-40% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w.

In some embodiments, the composition for pharmaceutical preparation comprises: semaglutide, about 10-20% w/w amino acids, about 20-50% N-(8-[2-hydroxybenzoyl]-amino)caprylic acid (NAC) or a salt thereof, and about 20-40% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w.

In some embodiments, the composition for pharmaceutical preparation comprises: semaglutide, about 10-20% w/w amino acids, about 20-55% N-(8-[2-hydroxybenzoyl]-amino)caprylic acid (NAC) or a salt thereof, and about 10-40% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 50-90% w/w; wherein the mass ratio of the N-(8-[2-hydroxybenzoyl]-amino) caprylic acid (NAC) or its salt to the medium-chain fatty acids or their salts is about 1:(0.5-2).

In some embodiments, the composition for pharmaceutical preparation comprises: semaglutide, about 10-20% w/w amino acids, about 20-55% N-(8-[2-hydroxybenzoyl]-amino)caprylic acid (NAC) or a salt thereof, and about 10-40% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w; wherein the mass ratio of the N-(8-[2-hydroxybenzoyl]-amino) caprylic acid (NAC) or its salt to the medium-chain fatty acids or their salts is about 1:(0.5-2).

In some embodiments, the composition for pharmaceutical preparation comprises: semaglutide, about 10-20% w/w amino acids, about 20-50% N-(8-[2-hydroxybenzoyl]-amino)caprylic acid (NAC) or a salt thereof, and no less than about 15% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w; wherein the mass ratio of the N-(8-[2-hydroxybenzoyl]-amino) caprylic acid (NAC) or its salt to the medium-chain fatty acids or their salts is about 1:(0.5-2).

In some embodiments, the composition for pharmaceutical preparation comprises: semaglutide, about 10-20% w/w amino acids, about 20-50% N-(8-[2-hydroxybenzoyl]-amino)caprylic acid (NAC) or a salt thereof, and no less than about 15% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w; wherein the mass ratio of the amino acid, N-(8-[2-hydroxybenzoyl]-amino) caprylic acid (NAC) or its salt, and medium-chain fatty acids or their salts is about (1-3):3:(1.5-6).

In some embodiments, the composition for pharmaceutical preparation comprises: semaglutide, about 10-20% w/w amino acids, about 20-50% N-(8-[2-hydroxybenzoyl]-amino)caprylic acid (NAC) or a salt thereof, and no less than about 15% w/w medium-chain fatty acids or salts thereof, wherein the total content of the amino acids, N-(8-[2-hydroxybenzoyl]-amino) caprylic acid (NAC) or a salt thereof and medium-chain fatty acids or salts thereof in the composition is about 70-80% w/w; wherein the mass ratio of the amino acid, N-(8-[2-hydroxybenzoyl]-amino) caprylic acid (NAC) or its salt, and medium-chain fatty acids or their salts is about 1:(2.5-3.5):(2.5-3.5).

In some embodiments, the composition for pharmaceutical preparation comprises: no more than 10% w/w semaglutide, about 10-20% w/w amino acids, about 20-50% N-(8-[2-hydroxybenzoyl]-amino)caprylic acid (NAC) or a salt thereof, and no less than about 15% w/w medium-chain fatty acids or salts thereof; wherein the total content of the amino acids, N-(8-[2-hydroxybenzoyl]-amino) caprylic acid (NAC) or a salt thereof and medium-chain fatty acids or salts thereof in the composition is about 70-80% w/w; wherein the mass ratio of the amino acid, N-(8-[2-hydroxybenzoyl]-amino) caprylic acid (NAC) or its salt, and medium-chain fatty acids or their salts is about 1:(2.5-3.5):(2.5-3.5).

In some embodiments, in the composition for pharmaceutical preparation, the medium-chain fatty acid is a fatty acid with a carbon chain length of 6 to 12 carbon atoms, and its structural formula is CH3(CH2)nCOOH, wherein n is greater than or equal to 4 and less than or equal to 10.

In some embodiments, in the composition for pharmaceutical preparation, the medium-chain fatty acid is selected from at least one of caproic acid, enanthic acid, caprylic acid, pelargonic acid, capric acid, undecanoic acid and lauric acid.

In some embodiments, in the composition for pharmaceutical preparation, the medium-chain fatty acid salt is selected from one or more of sodium salts, potassium salts and ammonium salts.

In some embodiments, the medium-chain fatty acid salt is sodium caprate.

In some embodiments, the amino acid is an amino acid comprising nitrogen in the side chain.

In some embodiments, the amino acid comprising nitrogen in the side chain is selected from at least one of lysine, arginine, asparagine, glutamine, tryptophan, ornithine, citrulline, pyrrolysine and histidine.

In some embodiments, the amino acid comprising nitrogen in the side chain is preferably selected from any one or more of citrulline and arginine.

In some embodiments, the N-(8-(2-hydroxybenzoyl) amino) caprylic acid (NAC) salt is selected from at least one of N-(8-(2-hydroxybenzoyl) amino) caprylic acid sodium salt, N-(8-(2-hydroxybenzoyl) amino) caprylic acid potassium salt and N-(8-(2-hydroxybenzoyl) amino) caprylic acid ammonium salt.

In some embodiments, the salt of NAC is sodium N-(8-(2-hydroxybenzoyl) amino) caprylate (SNAC).

Another aspect of the present disclosure provides a method for preparing an oral polypeptide tablet, the method comprising the step of using the composition for pharmaceutical preparation of the present disclosure to prepare the polypeptide tablet.

In some embodiments, the method comprising the step of using the composition for pharmaceutical preparation of the present disclosure to perform tableting.

In some embodiments, the method comprises the steps of: mixing granules A and granules B, and then tableting to obtain; wherein, the granules A comprise a polypeptide targeting the GLP-1 receptor; the granules B comprise one or more amino acids, N-(8-(2-hydroxybenzoyl) amino) caprylic acid (NAC) or a salt thereof, and one or more medium-chain fatty acids or salts thereof.

In some embodiments, the granules Aare granulated from a composition that comprises a polypeptide targeting GLP-1 receptor; and/or the granules B are granulated from a composition that comprises one or more amino acids, N-(8-(2-hydroxybenzoyl)amino)caprylic acid (NAC) or a salt thereof, and one or more medium-chain fatty acids or salts thereof.

In some embodiments, the granules Aare granulated from a composition that comprises a polypeptide targeting GLP-1 receptor and at least one excipient.

In some embodiments, the granules A comprise no less than about 10% w/w the polypeptide targeting GLP-1 receptor, such as 10% w/w, 20% w/w, 25% w/w, 30% w/w, 40% w/w, 42% w/w or 45% w/w.

In some embodiments, the granules A comprise no less than about 50% w/w excipients.

In some embodiments, the granules A comprise a polypeptide targeting GLP-1 receptor, fillers and binding agents.

In some embodiments, the granules B comprise one or more amino acids, N-(8-(2-hydroxybenzoyl)amino)caprylic acid (NAC) or a salt thereof, one or more medium-chain fatty acids or salts thereof, and at least one excipient.

In some embodiments, the granules B comprise one or more amino acids, N-(8-(2-hydroxybenzoyl)amino)caprylic acid (NAC) or a salt thereof, one or more medium-chain fatty acids or salts thereof, fillers, lubricants and disintegrants.

In some embodiments, the mass percentage of the amino acid, SNAC, and sodium caprate in granules B is at least about 60%.

In some embodiments, the mass percentage of the amino acid, SNAC, and sodium caprate in granules B is at least about 70%.

In some embodiments, the granules B comprise no less than about 10% w/w excipients.

In some embodiments, the granules B comprise no less than about 20% w/w excipients.

In some embodiments, the mass percentage of the granules A in the composition is no more than about 20% w/w.

In some embodiments, the mass percentage of the granules A in the composition is no more than about 10% w/w.

In some embodiments, the mass percentage of the granules B in the composition is at least about 80% w/w.

In some embodiments, the mass percentage of the granules B in the composition is at least about 90% w/w.

### Beneficial Effects

The oral polypeptide composition provided by the present disclosure significantly improves the absorption of polypeptide drugs by the gastrointestinal tract, thereby improving the efficacy of polypeptide drugs in the body.

Compared with Rybelsus®, under the same specifications, the oral semaglutide composition provided by the present disclosure significantly improves the oral bioavailability of semaglutide.

### BRIEF DESCRIPTION OF THE DRAWINGS

FIG. 1 is the cumulative dissolution-time curve of semaglutide tablets.

FIG. 2 is comparison of Cₘₐₓ for semaglutide tablets in beagle dogs.

FIG. 3 is comparison of Cₘₐₓ for semaglutide tablets prepared by different processes in beagle dogs.

FIG. 4 is comparison of Cₘₐₓ for different doses of semaglutide Tablets in cynomolgus monkeys.

FIG. 5 is detection and analysis of semaglutide concentration in beagle dog plasma after administration.

FIG. 6 is detection and analysis of teriparatide concentration in beagle dog plasma after administration.

FIG. 7 is detection and analysis of tirzepatide concentration in plasma after administration.

FIG. 8 is detection and analysis of octreotide concentration in plasma after administration.

### DETAILED DESCRIPTION OF THE EMBODIMENTS

According to the above content of this disclosure, in accordance with the common technical knowledge and conventional means in the art, without departing from the basic technical idea of this disclosure as mentioned above, various other forms of modifications, replacements or alterations can also be made.

### I. Definitions

Unless otherwise expressly indicated, throughout the specification and claims, the term “comprise” or its variants such as “include” or “comprising”, etc. will be understood to include the stated elements or components, without excluding other elements or other components.

The term “optional” or “optionally” means that the subsequent event or situation may or may not occur, and this description includes the occurrence or non-occurrence of the said event or situation.

As used herein, the term “about” means approximately, close to, roughly or around. When the term “about” is used in combination with a numerical range, it modifies the range by extending above and below the limits of the said numerical value. Generally, the term “about” is used herein to modify a value with a deviation of up to 20% above and below the value. In addition, especially for biological systems or processes, this term may mean up to one order of magnitude or up to five times the numerical value. Unless otherwise stated, when a specific value appears in this application and the claims, the meaning of “about” or “substantially including” should be assumed to be within the acceptable error range of the specific value.

### Oral Polypeptide Composition

As used herein, the terms “peptide”, “polypeptide” and “protein” are interchangeable, and these terms encompass both naturally occurring and non-naturally occurring polypeptides, as well as their fragments, mutants, derivatives and analogs as provided herein or as generally known in the art. The polypeptides also include polypeptides of drug molecules, nucleic acids encoding polypeptides, recombinant microorganisms for preparing polypeptides, vectors for expressing polypeptides, and polypeptides prepared using recombinant microorganisms.

In some embodiments, the polypeptide comprises at least 30, at least 40, at least 50 amino acid residues. In some embodiments, the polypeptide comprises 20-500 amino acid residues. In some embodiments, the polypeptide comprises one or more modified amino acid residues and/or non-protein amino acid residues.

In one embodiment, the polypeptide is a polypeptide targeting the glucagon-like peptide-1 (GLP-1) receptor.

As used herein, the term “polypeptide targeting the glucagon-like peptide-1 (GLP-1) receptor” refers to those polypeptides having a domain that binds to the GLP-1 receptor, able to selectively bind to the glucagon-like peptide-1 (GLP-1) receptor, thereby mimicking or enhancing the physiological functions of native GLP-1. In some embodiments, the amino acid sequence of the polypeptide targeting the glucagon-like peptide-1 (GLP-1) receptor may have at least 70%, 80%, 90% or higher sequence homology with native GLP-1 (such as human GLP-1(7-36)-amide), or be optimized for its stability, oral bioavailability or pharmacodynamic properties by modification (such as amino acid substitution, deletion or addition; fatty acid chain modification, PEGylation, cyclization, etc.).

In one embodiment, the polypeptide is semaglutide. In one embodiment, the oral polypeptide composition of the present disclosure contains 0.01-15.0% w/w of semaglutide, such as 0.2% w/w, 0.8% w/w, 0.9% w/w, 1.0% w/w, 1.5% w/w, 1.6% w/w, 1.7% w/w, 1.8% w/w, 3.3% w/w, 3.4% w/w, 6.9% w/w, 7.0% w/w, 8.0% w/w, 9.0% w/w, 10.0% w/w. Compared with Novo Nordisk's oral GLP-1 tablets, the oral polypeptide composition of the present disclosure has higher oral bioavailability for the same specification, lower semaglutide content when the oral bioavailability is the same, and less gastrointestinal side effects.

The term “pharmaceutically acceptable” refers to those compounds, materials, compositions and/or dosage forms that are suitable for contact with human and animal tissues within the scope of reasonable medical judgment without excessive toxicity, irritation, allergic reaction or other problems or complications commensurate with a reasonable benefit/risk ratio.

As used herein, the salts in the term “pharmaceutically acceptable salts” refer to water-soluble salts and water-insoluble salts, such as acetate, stilbenedisulfonate (4,4-diaminodiphenyl-2,2′-disulfonate), benzenesulfonate, benzoate, bicarbonate, bisulfate, bitartrate, borate, bromide, butyrate, calcium edetate, camphorsulfonate, carbonate, chloride, citrate, clavulanate, dihydrochloride, edetate, ethanedisulfonate, lauryl sulfate propionate, esilate, fumarate, glucoheptonate, gluconate, glutamate, glycouylarsanilate, hexafluorophosphate, hexylresorcinol salt, hydrabamine, hydrobromide, hydrochloride, hydroxynaphthoate, iodide, isothiosulfate, lactate, lactobionate, laurate, malate, maleate, mandelate, mesylate, methyl bromide, methyl nitrate, methyl sulfate, galactarate, naphthalenesulfonate, nitrate, ammonium N-methylglucosamine, 3-hydroxy-2-naphthoate, oleate, oxalate, palmitate, pamoate (1,1-methylene-bis-2-hydroxy-3-naphthoate, embonate), pantothenate, phosphate/diphosphate, picrate, polygalacturonate, propionate, p-toluenesulfonate, salicylate, stearate, basic acetate, succinate, sulfate, sulfosalicylate, suramate, tannate, tartrate, teoclate, toluenesulfonate, triethyl iodide and valerate.

As used herein, the term “absorption enhancer” refers to a class of excipients or compounds that can enhance the absorption efficiency of drugs in the gastrointestinal tract through physical, chemical or biological mechanisms, alone or in combination with other substances. In some embodiments, the absorption enhancer is a polypeptide-specific oral absorption enhancer.

The term “polypeptide-specific oral absorption enhancer” refers to a class of excipients or compounds that are specifically designed to enhance the oral absorption efficiency of polypeptide drugs. It should be noted that the absorption enhancer described herein has distinct characteristics from common excipients.

In some embodiments, the oral polypeptide composition of the present disclosure comprises an absorption enhancer no more than about 90% w/w, about 85% w/w, or about 80% w/w; and/or comprises an absorption enhancer no less than about 50% w/w, about 60% w/w, or about 70% w/w.

In some embodiments, the oral polypeptide composition of the present disclosure comprises about 70% w/w, 71% w/w, 72% w/w, 73% w/w, 74% w/w, 75% w/w, 76% w/w, 77% w/w, 78% w/w, 79% w/w, 80% w/w of an absorption enhancer.

In some embodiments, the absorption enhancer comprises one or more amino acids, N-(8-(2-hydroxybenzoyl) amino) caprylic acid (NAC) or its salts, and one or more medium-chain fatty acids or their salts.

In some embodiments, the absorption enhancer comprises an amino acid, N-(8-(2-hydroxybenzoyl) amino) caprylic acid (NAC) or its salts, and a medium-chain fatty acid or its salt.

In some embodiments, the oral polypeptide composition of the present disclosure comprises about 50% w/w, 49% w/w, 48% w/w, 47% w/w, 46% w/w, 45% w/w, 44% w/w, 43% w/w, 42% w/w, 41% w/w, 40% w/w of N-(8-(2-hydroxybenzoyl) amino) caprylic acid (NAC) or its salts; and/or no less than about 20% w/w, 21% w/w, 22% w/w, 23% w/w, 24% w/w, 25% w/w, 26% w/w, 27% w/w, 28% w/w, 29% w/w, 30% w/w of N-(8-(2-hydroxybenzoyl) amino) caprylic acid (NAC) or its salts.

In some embodiments, the oral polypeptide composition of the present disclosure comprises about 25% w/w, 26% w/w, 27% w/w, 28% w/w, 29% w/w, 30% w/w, 31% w/w, 32% w/w, 33% w/w, 34% w/w, 35% w/w, 36% w/w, 37% w/w, 38% w/w, 39% w/w, 40% w/w of N-(8-(2-hydroxybenzoyl) amino) caprylic acid (NAC) or its salts.

As used herein, the term “medium-chain fatty acid” refers to a class of straight-chain saturated monocarboxylic acids containing 6-12 carbon atoms, such as caproic acid, enanthic acid, caprylic acid, pelargonic acid, capric acid, undecanoic acid and lauric acid.

In some embodiments, the medium-chain fatty acid is a fatty acid with a carbon chain length of 6 to 12 carbon atoms, and its structural formula is CH₃(CH₂)nCOOH, where n is greater than or equal to 4 and less than or equal to 10.

In some embodiments, the oral polypeptide composition of the present disclosure comprises a medium-chain fatty acid or its salts no more than about 40% w/w, about 39% w/w, about 38% w/w, about 37% w/w, about 36% w/w or about 35% w/w; and/or comprises a medium-chain fatty acid or its salts not less than about 16% w/w, 18% w/w, 21% w/w, 22% w/w, 23% w/w, 24% w/w, 25% w/w, 26% w/w, 28% w/w, 29% w/w or 30% w/w.

In some embodiments, the oral polypeptide composition of the present disclosure comprises about 16% w/w, 17% w/w, 18% w/w, 19% w/w, 20% w/w, 21% w/w, 22% w/w, 23% w/w, 24% w/w, 25% w/w, 26% w/w, 28% w/w, 29% w/w, 30% w/w, 31% w/w, 32% w/w, 33% w/w, 34% w/w, 35% w/w of an medium-chain fatty acid or its salt.

As used herein, the term “amino acid” includes natural and non-natural amino acids. The term “natural amino acid” refers to 20 common amino acids, namely alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W) and tyrosine (Y). The term “non-natural amino acid” refers to an amino acid that is not naturally encoded or not found in the genetic code of any organism. For example, a non-natural amino acid can be a purely synthetic compound. Examples of non-natural amino acids include, but are not limited to, hydroxyproline, y carboxyglutamic acid, O-phosphoserine, azetidinecarboxylic acid, 2-aminoadipic acid, 3-aminoadipic acid, β-alanine, aminopropionic acid, 2-aminobutyric acid, 4-aminobutyric acid, 6-aminohexanoic acid, 2-aminoheptanoic acid, 2-amino-isobutyric acid, 3-amino-isobutyric acid, 2-aminoheptanedioic acid, tert-butylglycine, 2,4-diaminoisobutyric acid (Dap), desmosine, 2,2′-diaminoheptanedioic acid, 2,3-diaminopropionic acid (Dab), N-ethylglycine, N-methylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine (Orn), D-ornithine, D-arginine, p-aminophenylalanine, pentylglycine, pipecolic acid, and thioproline. In addition, this term also includes derivatives obtained by chemical modification of the C-terminal carboxyl group (or N-terminal amino group and/or its side-chain functional groups) of natural amino acids (or non-natural amino acids).

In some embodiments, the amino acid is an amino acid with a nitrogen-containing side chain. The term “amino acid with a nitrogen-containing side chain” refers to an amino acid in which the R group in the general formula H₂N—CH(R)—C(O)OH contains N, for example, the R group contains ═N⁺H₂, —NH, —NH—, —N═CH— etc. In some embodiments, the amino acid with a nitrogen-comprising side chain is selected from any one or more of lysine, arginine, asparagine, glutamine, tryptophan, ornithine, citrulline, pyrrolysine and histidine. In some embodiments, the amino acid is arginine.

In some embodiments, the oral polypeptide composition of the present disclosure comprises an amino acid no more than about 20% w/w, about 19% w/w, about 18% w/w, about 17% w/w, about 16% w/w, about 15% w/w; and/or comprises an amino acid not less than about 7% w/w, about 8% w/w, about 9% w/w, about 10% w/w.

In some embodiments, the oral polypeptide composition of the present disclosure comprises about 7.5% w/w, 7.8% w/w, 8.3% w/w, 8.5% w/w, 8.8% w/w, 9% w/w, 9.2% w/w, 9.5% w/w, 10% w/w, 10.5% w/w, 11% w/w, 11.5% w/w, 12% w/w, 12.5% w/w, 13% w/w, 13.5% w/w, 14% w/w, 14.5% w/w, 15% w/w of an amino acid.

As used herein, the term “excipient” refers to a substance added to a pharmaceutical composition to give a drug a certain shape, size or physical properties. Examples of excipients include, but are not limited to, carriers, fillers, binders, disintegrants, lubricants, glidants, taste masking agents and flavoring agents, stabilizers, dispersants, film formers, and preservatives. Suitable excipients can be selected based on factors such as the nature, type, route of administration, and therapeutic purpose of the drug. In some embodiments, the excipient is a filler. In some embodiments, the excipient is a filler, a disintegrant, and a lubricant. In some embodiments, the excipient is a filler, a disintegrant, a lubricant, and a binder.

In some embodiments, the oral polypeptide composition of the present disclosure includes no more than about 30% w/w, 29% w/w, 28% w/w, 27% w/w, 26% w/w, 25% w/w of excipients; and/or, comprises no less than about 10% w/w, 11% w/w, 12% w/w, 13% w/w, 14% w/w, 15% w/w of excipients. In some embodiments, the oral polypeptide composition of the present disclosure includes about 15% w/w, 16% w/w, 17% w/w, 18% w/w, 19% w/w, 20% w/w, 21% w/w, 22% w/w, 23% w/w, 24% w/w, 25% w/w of excipients.

The term “filler” refers to an excipient used to increase the weight and volume of the tablet and facilitate tableting. Examples of fillers include, but are not limited to, starch, powdered sugar, dextrin, lactose, microcrystalline cellulose, pregelatinized starch, mannitol, calcium sulfate, and the like. In one embodiment, the filler is micro-crystalline cellulose.

The term “disintegrant” refers to an excipient that helps tablets break into fine particles in gastrointestinal fluid. Examples of disintegrants include, but are not limited to, dry starch, sodium carboxymethyl cellulose, low-substituted carboxymethyl cellulose, cross-linked polyvinyl pyrrolidone, cross-linked sodium carboxymethyl cellulose, sodium carboxymethyl starch. In one embodiment, the disintegrant is sodium carboxymethyl starch.

The term “lubricant” refers to an excipient used to reduce friction between particles, prevent raw materials from adhering to the punch surface, or reduce friction between tablets and the wall of the die hole. Examples of lubricants include, but are not limited to, magnesium stearate, micro-powdered silica gel, talc, polyethylene glycol, sodium lauryl sulfate, and the like. In one embodiment, the lubricant is sodium lauryl sulfate.

The terms “binding agent”, “adhesive” or “binder” are used interchangeably and refer to viscous substances added to solve the problem that the raw material itself has low viscosity and is difficult to granulate, such as polyvinyl pyrrolidone (povidone). In one embodiment, the binder is povidone.

The term “solid dosage form” or “solid preparation” refers to solid pharmaceutical preparation with strong cohesion and shape stability. The form of solid preparations includes but is not limited to tablets, capsules, granules, powders, pellets, and pills. Solid preparations can be ordinary preparations, sustained-release or rapid-release preparations (such as osmotic pump tablets, sustained-release tablets, sustained-release capsules, rapidly disintegrating tablets, etc.). In some embodiments, the pharmaceutical composition of the present disclosure is a tablet, capsule or granule. In some embodiments, the pharmaceutical composition of the present disclosure is a tablet.

The term “tablet” refers to a round or irregular sheet-like solid preparation made of raw drug and suitable excipients. Tablets are mainly oral ordinary tablets (including sugar-coated tablets and film-coated tablets), and there are also lozenges, sublingual tablets, oral patches, chewable tablets, dispersible tablets, soluble tablets, effervescent tablets, vaginal tablets, vaginal effervescent tablets, sustained-release tablets, controlled-release tablets, enteric-coated tablets (including enteric-coated tablets and colon-localized enteric-coated tablets) and orodisintegrating tablets. The tablets disclosed in the present disclosure can be coated by methods well known in the art.

The term “specification” refers to the amount of drug ingredients contained in a unit preparation drug, usually expressed in terms of weight or content per tablet, per pill, per tube, etc. It includes indicators such as the content, potency or loading of the main drug.

In some embodiments, the specification of the oral polypeptide tablet is 1-50 mg, such as 1 mg, 2 mg, 3 mg, 4 mg, 5 mg, 6 mg, 6.5 mg, 6.8 mg, 7 mg, 7.1 mg, 7.4 mg, 7.5 mg, 7.9 mg, 8 mg, 8.6 mg, 9 mg, 10 mg, 14 mg, 15 mg, 16 mg, 17 mg, 18 mg, 19 mg, 20 mg, 21 mg, 22 mg, 23 mg, 24 mg, 25 mg, 26 mg, 28 mg, 30 mg, 31 mg, 32 mg, 33 mg, 34 mg, 35 mg, 40 mg, 50 mg.

The term “tablet weight” refers to the weight of a single tablet. In some embodiments, the tablet weight of the oral polypeptide tablet is 300-1000 mg, such as 300 mg, 350 mg, 400 mg, 450 mg, 460 mg, 470 mg, 480 mg, 490 mg, 500 mg, 510 mg, 520 mg, 530 mg, 540 mg, 550 mg, 600 mg, 800 mg.

The term “unit preparation” or “unit dosage form” refers to the smallest dosing unit or the smallest packaging unit that can be split in a drug, such as tablets, capsules, powders, injections, capsules in capsules, micro-patch systems in capsules, etc. These units can exist independently physically and can be precisely divided in dosage. Unit preparations usually comprise a predetermined amount of active ingredients to produce the desired therapeutic effect and are used in combination with appropriate pharmaceutical excipients.

As used herein, the term “capsule in capsule” is a special delivery system for multi-stage release formulations. After the release conditions are reached, the outer capsule releases first and the inner capsule releases later.

As used herein, the term “micro-patch system in capsule” means that after the capsule enters the gastrointestinal tract, the capsule shell dissolves and the micro-patch is released. The patch can adhere to the wall of the gastrointestinal tract to increase the absorption efficiency of the drug by locally releasing the drug.

The term “single-agent formulation” refers to a single dosage form comprises only one drug or active ingredient in the prescription. In some embodiments, the oral polypeptide composition is a single-agent formulation.

### Indications

The term “treating” includes inhibiting, alleviating, preventing or eliminating one or more symptoms or side effects associated with the disease, condition or disorder being treated.

The term “effective amount” or “therapeutically effective amount” refers to a dose sufficient to treat, inhibit or alleviate one or more symptoms of the disease state being treated or otherwise provide the desired pharmacological and/or physiological effect. The exact dose will vary depending on a variety of factors, such as subject-dependent variables (e.g., age, immune system health, etc.), the disease or illness, and the treatment being administered. The effect of an effective amount can be relative to a control. Such controls are known in the art and discussed herein, and can be, for example, the condition of the subject before or without administration of the drug or drug combination, or in the case of a drug combination, the combined effect can be compared to the effect of administering only one drug.

Indications for the oral polypeptide composition disclosed herein include but are not limited to endocrine diseases, neurodegenerative diseases, vascular diseases, kidney diseases, fatty liver diseases, and cancer, polycystic ovary syndrome, bone metabolism and osteoporosis, knee osteoarthritis, sleep apnea syndrome, osteoarthritis, acromegaly, carcinoid syndrome, islet cell tumors, intestinal fistulas, esophageal varicose bleeding, and metabolic muscle diseases. In some embodiments, the endocrine diseases comprise glucose metabolism disorders, diabetes, and obesity.

In some embodiments, the neurodegenerative diseases comprise Alzheimer's disease and other forms of dementia, Parkinson's disease, multiple sclerosis, and Huntington's disease. In some embodiments, the cardiovascular diseases comprise atherosclerosis, hyper-coagulable state and hypocoagulable state, coronary artery disease, and cerebrovascular events. In some embodiments, the kidney diseases comprise renal failure. In some embodiments, the fatty liver comprises non-alcoholic fatty liver.

In some embodiments, the polypeptide composition in the present disclosure is semaglutide, and the indication is selected from at least one of type 1 diabetes, type 2 diabetes, obesity, cardiovascular disease, non-alcoholic fatty liver disease, chronic kidney disease, Alzheimer's disease, cancer, polycystic ovary syndrome, metabolic syndrome, bone metabolism and osteoporosis, knee osteoarthritis, sleep apnea syndrome, osteoarthritis and metabolic muscle disease and etc.

### Composition for Pharmaceutical Preparation and Tablet Preparation Method

The composition for pharmaceutical preparation disclosed herein refers to a composition for preparing an oral polypeptide tablet, which comprises granules A and granules B, wherein granules A comprises polypeptide molecules but do not comprise an absorption enhancer; granules B comprises an absorption enhancer but do not comprise polypeptide molecules, wherein the absorption enhancer comprises one or more amino acids, N-(8-(2-hydroxybenzoyl) amino) caprylic acid (NAC) or its salt, and one or more medium-chain fatty acids or their salts. In some embodiments, granules A comprises polypeptide molecules and excipients but do not comprise an absorption enhancer; and/or, granules B comprises an absorption enhancer and excipients but do not comprise polypeptide molecules.

In the composition for pharmaceutical preparation of the present disclosure, particles A and particles B can be produced by methods known to those skilled in the art, for example, by using wet granulation methods known for the production of “built-up” granules or “broken-down” granules. Methods for the formation of built-up granules can be operated continuously. For example, they may involve simultaneously spraying the granulation mass with granulation solution and drying, which can be carried out in a drum granulator, pan granulators, disc granulators, a fluidized bed through spray-drying or spray-solidifying. Or they can be operated discontinuously, such as in a fluidized bed, a rotary fluid bed, a batch mixer (like a high shear mixer or a low shear mixer), or a spray-drying drum. Methods for the production of broken-down granules, which may be carried out discontinuously and in which the granulation mass first forms a wet aggregate with the granulation solution, which is subsequently comminuted or by other means formed into granules of the desired size and the granules may then be dried. Suitable equipment for the granulation step are planetary mixers, low shear mixers, high shear mixers, extruders and spheronizers. Granules may be also formed by dry granulation techniques in which the pharmaceutically active agent is compressed with the excipients to form relatively large moldings, for example slugs or ribbons, which are comminuted by grinding, and the ground material serves as the tabletting material to be later compacted. Suitable equipment for dry granulation is roller compaction equipment from Gerteis.

The method of preparing oral polypeptide tablets using the composition for pharmaceutical preparation of the present disclosure is also well known to those skilled in the art. To compact the tabletting material into a solid oral dosage form, for example a tablet, a tablet press may be used. In a tabletting press, the tabletting material is filled (e.g. force fed or gravity fed) into a die cavity. The tabletting material is then compacted by a punch with pressure. Subsequently, the resulting compact, or tablet is ejected from the tabletting press. The above-mentioned compaction process is subsequently referred to herein as the “compaction process”. Suitable tablet presses include, but are not limited to, rotary tablet presses and eccentric tablet presses.

The process for preparing the oral polypeptide tablets disclosed herein is not limited thereto, for example, tablets can be obtained by direct compression. The oral polypeptide tablets prepared using the composition for pharmaceutical preparation disclosed herein can significantly improve the oral bioavailability of the tablets compared to the polypeptide tablets obtained by direct compression when the composition is the same.

### II. Example

The present disclosure is further explained below with reference to examples. The description of specific exemplary embodiments of the present disclosure is for the purpose of illustration and demonstration. These descriptions are not intended to confine the present disclosure to the exact form disclosed, and it is evident that numerous modifications and variations can be made in accordance with the teachings of the present disclosure's specification. The purpose of selecting and describing the exemplary embodiments is to explain the specific principles of the present disclosure and its practical application, so that those skilled in the art can implement and utilize various different exemplary embodiments of the present disclosure and various different selections and changes.

The experimental methods used in the following examples are conventional methods unless otherwise specified.

The materials, reagents, etc. used in the following examples can be obtained from commercial channels unless otherwise specified.

The specific embodiments of the present disclosure are further described below. It should be noted that the description of these embodiments is used to help understand the present disclosure, but does not constitute a limitation of the present disclosure. In addition, the technical features involved in the embodiments described below can be combined with each other as long as they do not conflict with each other.

The experimental methods in the following examples are conventional methods unless otherwise specified, and the experimental materials used in the following examples can be purchased through conventional commercial channels unless otherwise specified.

Unless otherwise stated, the amino acids used in the specific examples below are in the L conformation.

### Example 1: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.09% w/w semaglutide, 10.87% w/w arginine, 32.61% w/w SNAC, 32.61% w/w sodium caprate, 19.82% w/w microcrystalline cellulose, 1% w/w sodium lauryl sulfate, 2% w/w sodium carboxymethyl starch. Semaglutide:SNAC:sodium caprate:arginine=5:150:150:50
2) Tableting: After mixing, the material is tableted. A flat punch die with a diameter of 10 mm is used for tableting, and 460 mg of material is fed per tablet. The tablet was taken out from the mold to obtain a 10 mm round flat tablet with 5 mg semaglutide.

### Example 2: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.09% w/w semaglutide, 10.87% w/w arginine, 43.48% w/w SNAC, 21.74% w/w sodium caprate, 19.82% w/w microcrystalline cellulose, 1% w/w sodium lauryl sulfate, 2% w/w sodium carboxymethyl starch. Semaglutide:SNAC:sodium caprate:arginine=5:200:100:50
2) Tableting: After mixing, the material is tableted. A flat punch die with a diameter of 10 mm is used for tableting, and 460 mg of material is fed per tablet. The tablet was taken out from the mold to obtain a 10 mm round flat tablet with 5 mg semaglutide.

### Example 3: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.09% w/w semaglutide, 10.87% w/w arginine, 54.35% w/w SNAC, 10.87% w/w sodium caprate, 19.82% w/w microcrystalline cellulose, 1% w/w sodium lauryl sulfate, 2% w/w sodium carboxymethyl starch. Semaglutide:SNAC:sodium caprate:arginine=5:250:50:50
2) Tableting: After mixing, the material is tableted. A flat punch die with a diameter of 10 mm is used for tableting, and 460 mg of material is fed per tablet. The tablet was taken out from the mold to obtain a 10 mm round flat tablet with 5 mg semaglutide.

### Example 4: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 0.98% w/w semaglutide, 29.41% w/w arginine, 29.41% w/w SNAC, 29.41% w/w sodium caprate, 8.08% w/w microcrystalline cellulose, 0.9% w/w sodium lauryl sulfate, 1.8% w/w sodium carboxymethyl starch. Semaglutide:SNAC:sodium caprate:arginine=5:150:150:150
2) Tableting: After mixing, the material is tableted. A flat punch die with a diameter of 10 mm is used for tableting, and 510 mg of material is fed per tablet. The tablet was taken out from the mold to obtain a 10 mm round flat tablet with 5 mg semaglutide.

### Example 5: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.09% w/w semaglutide, 13.04% w/w arginine, 39.13% w/w SNAC, 39.13% w/w sodium caprate, 4.61% w/w microcrystalline cellulose, 1% w/w sodium lauryl sulfate, 2% w/w sodium carboxymethyl starch. Semaglutide:SNAC:sodium caprate:arginine=5:180:180:60
2) Tableting: After mixing, the material is tableted. A flat punch die with a diameter of 10 mm is used for tableting, and 460 mg of material is fed per tablet. The tablet was taken out from the mold to obtain a 10 mm round flat tablet with 5 mg semaglutide.

### Example 5: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.09% w/w semaglutide, 5.43% w/w arginine, 32.61% w/w SNAC, 32.61% w/w sodium caprate, 25.26% w/w microcrystalline cellulose, 1% w/w sodium lauryl sulfate, 2% w/w sodium carboxymethyl starch. Semaglutide:SNAC:sodium caprate:arginine=5:150:150:25
2) Tableting: After mixing, the material is tableted. A flat punch die with a diameter of 10 mm is used for tableting, and 460 mg of material is fed per tablet. The tablet was taken out from the mold to obtain a 10 mm round flat tablet with 5 mg semaglutide.

### Comparative Example 1: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.09% w/w semaglutide, 10.87% w/w arginine, 65.22% w/w sodium caprate, 19.82% w/w microcrystalline cellulose, 1% w/w sodium lauryl sulfate, 2% w/w sodium carboxymethyl starch. Semaglutide:sodium caprate:arginine=5:300:50
2) Tableting: After mixing, the material is tableted. A flat punch die with a diameter of 10 mm is used for tableting, and 460 mg of material is fed per tablet. The tablet was taken out from the mold to obtain a 10 mm round flat tablet with 5 mg semaglutide.

### Comparative Example 2: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.09% w/w semaglutide, 10.87% w/w arginine, 65.22% w/w SNAC, 19.82% w/w microcrystalline cellulose, 1% w/w sodium lauryl sulfate, 2% w/w sodium carboxymethyl starch. Semaglutide:SNAC:arginine=5:300:50
2) Tableting: After mixing, the material is tableted. A flat punch die with a diameter of 10 mm is used for tableting, and 460 mg of material is fed per tablet. The tablet was taken out from the mold to obtain a 10 mm round flat tablet with 5 mg semaglutide.

### Comparative Example 3: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.09% w/w semaglutide, 32.61% w/w SNAC, 32.61% w/w sodium caprate, 30.69% w/w microcrystalline cellulose, 1% w/w sodium lauryl sulfate, 2% w/w sodium carboxymethyl starch. Semaglutide:SNAC:sodium caprate=5:150:150
2) Tableting: After mixing, the material is tableted. A flat punch die with a diameter of 10 mm is used for tableting, and 460 mg of material is fed per tablet. The tablet was taken out from the mold to obtain a 10 mm round flat tablet with 5 mg semaglutide.

### Comparative Example 4: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.09% w/w semaglutide, 65.22% w/w sodium caprate, 30.69% w/w microcrystalline cellulose, 1% w/w sodium lauryl sulfate, 2% w/w sodium carboxymethyl starch. Semaglutide:sodium caprate=5:300
2) Tableting: After mixing, the material is tableted. A flat punch die with a diameter of 10 mm is used for tableting, and 460 mg of material is fed per tablet. The tablet was taken out from the mold to obtain a 10 mm round flat tablet with 5 mg semaglutide.

### Comparative Example 5: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.09% w/w semaglutide, 65.22% w/w SNAC, 30.69% w/w microcrystalline cellulose, 1% w/w sodium lauryl sulfate, 2% w/w sodium carboxymethyl starch. Semaglutide:SNAC=5:300
2) Tableting: After mixing, the material is tableted. A flat punch die with a diameter of 10 mm is used for tableting, and 460 mg of material is fed per tablet. The tablet was taken out from the mold to obtain a 10 mm round flat tablet with 5 mg semaglutide.

### Comparative Example 6: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.09% w/w semaglutide, 10.87% w/w arginine, 85.04% w/w microcrystalline cellulose, 1% w/w sodium lauryl sulfate, 2% w/w sodium carboxymethyl starch. Semaglutide:arginine=5:50
2) Tableting: After mixing, the material is tableted. A flat punch die with a diameter of 10 mm is used for tableting, and 460 mg of material is fed per tablet. The tablet was taken out from the mold to obtain a 10 mm round flat tablet with 5 mg semaglutide.

### Comparative Example 7: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.09% w/w semaglutide, 10.87% w/w arginine, 10.87% w/w SNAC, 54.35% w/w sodium caprate, 19.82% w/w microcrystalline cellulose, 1% w/w sodium lauryl sulfate, 2% w/w sodium carboxymethyl starch. Semaglutide:SNAC:sodium caprate:arginine=5:50:250:50
2) Tableting: After mixing, the material is tableted. A flat punch die with a diameter of 10 mm is used for tableting, and 460 mg of material is fed per tablet. The tablet was taken out from the mold to obtain a 10 mm round flat tablet with 5 mg semaglutide.

### Comparative Example 8: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.09% w/w semaglutide, 21.74% w/w arginine, 32.61% w/w SNAC, 32.61% w/w sodium caprate, 8.96% w/w microcrystalline cellulose, 1% w/w sodium lauryl sulfate, 2% w/w sodium carboxymethyl starch. Semaglutide:SNAC:sodium caprate:arginine=5:150:150:100
2) Tableting: After mixing, the material is tableted. A flat punch die with a diameter of 10 mm is used for tableting, and 460 mg of material is fed per tablet. The tablet was taken out from the mold to obtain a 10 mm round flat tablet with 5 mg semaglutide.

### Comparative Example 9: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.09% w/w semaglutide, 6.52% w/w arginine, 19.57% w/w SNAC, 19.57% w/w sodium caprate, 50.25% w/w microcrystalline cellulose, 1% w/w sodium lauryl sulfate, 2% w/w sodium carboxymethyl starch. Semaglutide:SNAC:sodium caprate:arginine=5:90:90:30
2) Tableting: After mixing, the material is tableted. A flat punch die with a diameter of 10 mm is used for tableting, and 460 mg of material is fed per tablet. The tablet was taken out from the mold to obtain a 10 mm round flat tablet with 5 mg semaglutide.

### Comparative Example 10: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.09% w/w semaglutide, 95.91% w/w microcrystalline cellulose, 1% w/w sodium lauryl sulfate, 2% w/w sodium carboxymethyl starch.
2) Tableting: After mixing, the material is tableted. A flat punch die with a diameter of 10 mm is used for tableting, and 460 mg of material is fed per tablet. The tablet was taken out from the mold to obtain a 10 mm round flat tablet with 5 mg semaglutide.

### Test Example 1

The dissolution and release rate were determined according to the dissolution and release rate determination method (Chinese Pharmacopoeia 2020 Edition Part IV General Rules 0931 Second Method). The specific method is as follows: 500 ml of 0.05% Brij® solution-pH6.8 phosphate buffer (weigh 6.8 g of anhydrous potassium dihydrogen phosphate, 0.896 g of sodium hydroxide and 0.5 g of Brij®, add 1000 ml of water to dissolve, and adjust the pH to 6.8 with phosphoric acid or sodium hydroxide) is used as the dissolution medium, and the rotation speed is 70 revolutions per minute. At 0 min, the samples prepared in Example 1 and Comparative Examples 3-5 were put into each group of 3 tablets, and samples were taken at 5 min, 10 min, 15 min, 20 min, 30 min, 45 min, and 60 min. After the 60 min sampling, the dissolution cup was subjected to 5-minute ultrasonic treatment for sampling. For the test solution, take 4.5 ml of the dissolution solution, filter it, and collect the subsequent filtrate (if the initial 3 ml of the filtrate is turbid, centrifuge it). Reference solution: Take an appropriate amount of semaglutide reference, weigh accurately, dissolve with dissolution medium and quantitatively dilute to make a solution containing about 32 g of semaglutide per 1 ml.

The injection volume of the reference solution is 25 μl, and the injection volume of the test solution is 100 μl (4 mg specification), 50 μl (8 mg specification), and 25 μl (16 mg specification). Octadecylsilane bonded silica gel was used as filler (Phenomenex Kinetex C18, 150×4.6 mm, 2.6 m or chromatographic column with equivalent performance); 0.1 mol/L diammonium hydrogen phosphate buffer (weigh 23.76 g diammonium hydrogen phosphate, add appropriate amount of water to completely dissolve, add water to 1800 ml, adjust pH to 3.6 with concentrated phosphoric acid, and filter)-acetonitrile (9:1) was used as mobile phase A, acetonitrile-isopropanol-water (3:1:1) was used as mobile phase B, and linear gradient elution was performed according to the table below; the detection wavelength was 215 nm; the column temperature was 35° C.; the injection plate temperature was 6° C.; the flow rate was 0.5 ml per minute; the injection volume was 10 μl.

The sample was tested using an Agilent 1260 InfinityII high performance liquid chromatograph. The injection volume of the reference solution was 25 μl, and the injection volume of the test solution was 801. Use octadecylsilane bonded silica gel as filler (Phenomenex Kinetex C18, 150×4.6 mm, 2.6 m or chromatographic column with equivalent performance); use 0.1 mol/L diammonium hydrogen phosphate buffer (weigh 23.76 g of diammonium hydrogen phosphate, add appropriate amount of water to completely dissolve it, add water to 1800 ml, adjust the pH to 3.6 with concentrated phosphoric acid, and filter)-acetonitrile (9:1) as mobile phase A, and acetonitrile-isopropanol-water (3:1:1) as mobile phase B, and perform linear gradient elution according to Table 1; the detection wavelength is 215 nm; the column temperature is 35° C.; the injection plate temperature is 6° C.; the flow rate is 0.5 ml per minute.

TABLE 1

| Time (min) | Mobile phase A (%) | Mobile phase B (%) |
|---|---|---|
| 0 | 47 | 53 |
| 14 | 47 | 53 |
| 14.1 | 0 | 100 |
| 16 | 0 | 100 |
| 16.1 | 47 | 53 |
| 20 | 47 | 53 |

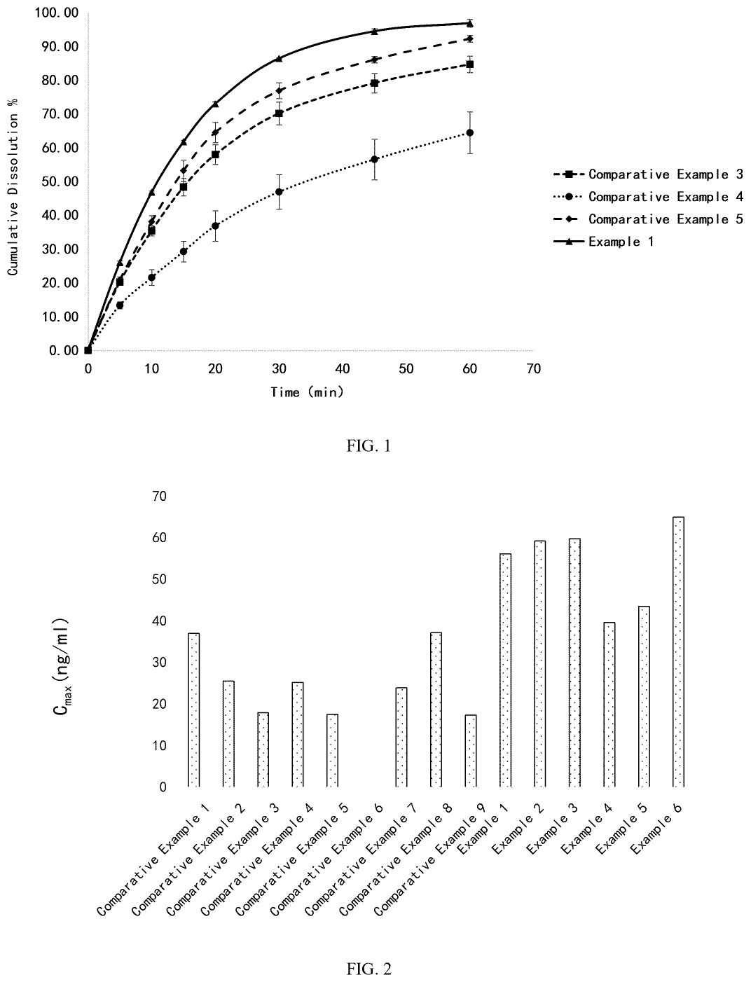

The dissolution test results of the above semaglutide tablets are shown in Table 2 and FIG. 1. The results show that when the prescription contains SNAC, sodium caprate and arginine at the same time, the dissolution rate of semaglutide is the fastest.

TABLE 2

| Dissolution time (min) | | 0 | 5 | 10 | 15 | 20 | 30 | 45 | 60 |
|---|---|---|---|---|---|---|---|---|---|
| Comparative | Cumulative | 0.00 | 20.22 | 35.39 | 48.35 | 57.97 | 70.15 | 79.13 | 84.67 |
| Example 3 | dissolution | | | | | | | | |
| Comparative | (%) | 0.00 | 13.36 | 21.57 | 29.26 | 36.84 | 46.92 | 56.55 | 64.45 |
| Example 4 | | | | | | | | | |
| Comparative | | 0.00 | 20.84 | 38.09 | 53.19 | 64.52 | 76.86 | 86.06 | 92.27 |
| Example 5 | | | | | | | | | |
| Example 1 | | 0.00 | 26.04 | 46.83 | 61.78 | 73.01 | 86.44 | 94.48 | 96.91 |

### Test Example 2

The absorption of the semaglutide tablets prepared above after oral administration in beagle dogs was tested. The specific method is: the experimental animals (Weight 10-12 kg) need to fast for 12 hours and deprive of water for 6 hours before administration, and the fasting and deprivation of water are lifted 1 hour after administration. Before administration, 1 ml of blank blood is collected using an EDTA-K2 blood collection tube as the blood sample at 0 o'clock in the experiment. The tablets prepared in Examples 1-5 and Comparative Examples 1-9 are orally administered to beagle dogs by gavage and 15 ml of water is given at the same time to ensure successful swallowing. Each group consisted of 5 animals, all of which were male. The mean body weight of each group was ensured to be the same. Subsequently, blood is collected from the forearm vein of the beagle dogs using an EDTA-K2 blood collection tube at the following time points after administration: 0.5 h, 1 h, 2 h, and 3 h, and the blood volume at each blood collection point is 1 ml. The whole blood of the beagle dogs is collected and centrifuged. After centrifugation, the supernatant plasma blood sample is taken and frozen at −20° C. for subsequent mass spectrometry analysis. The mass spectrometry detection was implemented after verification according to the method described in the public document “Quantitative Analysis of Semaglutide and Tirzepatide in Rat Plasma by SCIEX LC-MS/MS System”.

The absorption test results of the above-mentioned semaglutide tablets after oral administration to beagle dogs are shown in Table 3 and FIG. 2 below.

TABLE 3

| | Cₘₐₓ (ng/ml) |
|---|---|
| NO. | Mean |
| Comparative Example 1 | 36.96 |
| Comparative Example 2 | 25.54 |
| Comparative Example 3 | 17.94 |
| Comparative Example 4 | 25.22 |
| Comparative Example 5 | 17.52 |
| Comparative Example 6 | 0 |
| Comparative Example 7 | 23.9 |
| Comparative Example 8 | 37.12 |
| Comparative Example 9 | 17.34 |
| Comparative Example 10 | 0 |
| Example 1 | 56.14 |
| Example 2 | 59.2 |
| Example 3 | 59.74 |
| Example 4 | 39.64 |
| Example 5 | 43.5 |
| Example 6 | 64.94 |

### Example 7: Preparation of Semaglutide Tablets

1) Mixing: Prepare the powder of the drug preparation system by taking the raw materials. After the powder of each component is sieved through a 40-mesh sieve, the components are accurately weighed according to the mass ratio shown below. After weighing, the components are mixed using a three-dimensional mixer (speed 50 rpm, time 300 s).

The formulation of the semaglutide pharmaceutical composition is as follows: 1.74% w/w semaglutide, 10.870% w/w arginine, 32.61% w/w SNAC, 32.61% w/w sodium caprate, 19.17% w/w microcrystalline cellulose, 10% w/w sodium lauryl sulfate, 20% w/w sodium carboxymethyl starch. Semaglutide:SNAC:sodium caprate:arginine=8:150:150:50
2) Tablet pressing: After mixing, the material is taken for tablet pressing. A 13.5*7.5 mm shallow arc special-shaped punch is used for tablet pressing. Each tablet is fed with 460 mg of material. The tablet is taken out from the mold to obtain an oval tablet with 8 mg semaglutide.

### Example 8: Preparation of Semaglutide Tablets

1) Mixing: Prepare the drug preparation system powder from the raw materials. After passing the powder of each component through a 40-mesh sieve, accurately weigh each component according to the mass configuration ratio shown below. After weighing, transfer it into a PE bag and mix it 1-2 times, shaking 100-200 times/time.

Material A: 10.53% w/w semaglutide, 68.42% w/w microcrystalline cellulose, 21.05% w/w povidone K90

Material B: 11.38% w/w arginine, 34.12% w/w SNAC, 34.12% w/w sodium caprate, 17.24% w/w microcrystalline cellulose, 1.05% w/w sodium lauryl sulfate, 2.09% w/w sodium carboxymethyl starch
2) Granulation: Set the cooling water temperature of the dry granulator The temperature is 10-25° C., the roller pressure is 70±30 bar, the feeding speed is 20-60 rpm, the roller speed is 6-12 rpm, the granulation speed is 60-100 rpm, and the screen specification is 16/20 mesh; the equipment is turned on to conduct trial granulation of material A. If it does not meet the requirements, the equipment parameters are appropriately adjusted and trial production is carried out again. If it meets the requirements, formal granulation is carried out. The actual parameters are recorded during the granulation process until all the powders are granulated to obtain granules A.

Granulate material B using the same process to obtain granules B.
3) Tabletting: According to the mass configuration ratio of 14.67% w/w granules A, 84.83% w/w granules B, and 0.5% w/w magnesium stearate, each component is accurately weighed, and after weighing, it is transferred into a PE bag and mixed 1-2 times, and shaken 100-200 times/time. After mixing, the material was taken out for tableting, and a 13.5*7.5 mm shallow arc special-shaped punch was used for tableting, and 518.2 mg of material was added to each tablet. The tablet was taken out from the mold to obtain an oval tablet with 8 mg semaglutide.

### Example 9: Preparation of Semaglutide Tablets

1) Mixing: Prepare the drug preparation system powder by taking the raw materials. After passing the powder of each component through a 40-mesh sieve, accurately weigh each component according to the mass configuration ratio shown below. After weighing, transfer it into a PE bag and mix it 1-2 times, shaking 100-200 times/time.

Material A: 42.11% w/w semaglutide, 36.84% w/w microcrystalline cellulose, 21.05% w/w povidone K90

Material B: 11.38% w/w arginine, 34.12% w/w SNAC, 34.12% w/w sodium caprate, 17.24% w/w microcrystalline cellulose, 1.05% w/w sodium lauryl sulfate, 2.09% w/w sodium carboxymethyl starch
2) Granulation: The dry granulator is set with cooling water temperature of 10-25° C., roller pressure of 70±30 bar, feeding speed of 20-60 rpm, roller speed of 6-12 rpm, granulation speed of 60-100 rpm, and screen specification of 16/20 mesh; the equipment is turned on to conduct trial granulation of material A. If it does not meet the requirements, the equipment parameters are appropriately adjusted and trial production is carried out again. If it meets the requirements, formal granulation is carried out. The actual parameters are recorded during the granulation process until all the powders are granulated to obtain granules A.

Granulate material B using the same process to obtain granules B.
3) Tableting: According to the mass configuration ratio of 4.12% w/w granule A, 95.36% w/w granule B, and 0.52% w/w magnesium stearate, each component is accurately weighed, and after weighing, it is transferred into a PE bag and mixed 1-2 times, and shaken 100-200 times/time. After mixing, the material is taken for tableting. A 13.5*7.5 mm shallow arc special-shaped punch is used for tableting, and 461 mg of material is added per tablet. The tablet is taken out of the mold to obtain an oval tablet with 8 mg semaglutide.

### Test Example 3

The absorption of the semaglutide tablets prepared above was tested after oral administration in beagle dogs. The specific method is as follows: the experimental animals (Weight 10-12 kg) were fasted for 12 hours and deprived of water for 6 hours before administration, and the fasting and deprivation of water were lifted 1 hour after administration. Before administration, 1 ml of blank blood was collected using an EDTA-K2 blood collection tube as the blood sample at 0 o'clock in the experiment. The tablets prepared in Examples 7-9 were orally administered to beagle dogs by gavage and 15 ml of water was given at the same time to ensure successful swallowing. Each group consisted of 5 animals, all male, and the mean weight of each group was ensured to be the same. Subsequently, blood was collected from the forearm vein of the beagle dogs using an EDTA-K2 blood collection tube at the following time points after administration: 0.5 h, 1 h, 2 h, and 3 h, and the blood volume at each blood collection point was 1 ml. The whole blood collected from the beagle dogs was centrifuged, and the supernatant plasma blood sample was taken after centrifugation and frozen at −20° C. for subsequent mass spectrometry analysis. The mass spectrometry detection was implemented after verification according to the method described in the public document “Quantitative Analysis of Semaglutide and Tirzepatide in Rat Plasma by SCIEX LC-MS/MS System”.

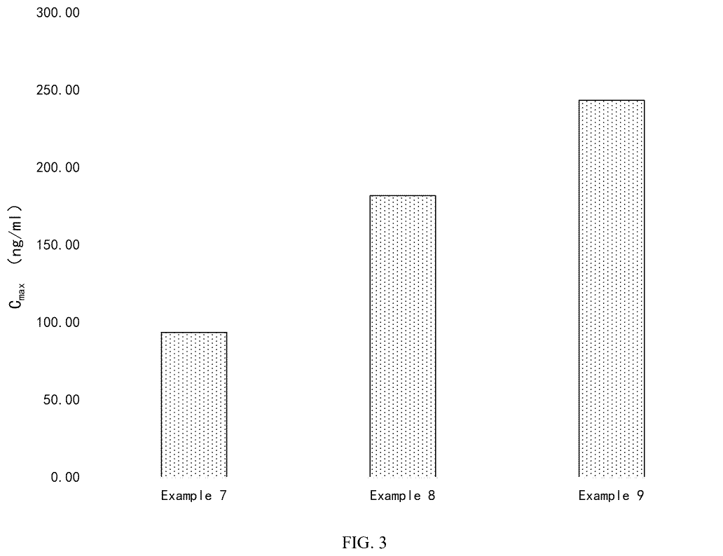

The absorption test results of the above-mentioned semaglutide tablets after oral administration to beagle dogs are shown in Table 4 and FIG. 3 below.

TABLE 4

| | Cₘₐₓ (ng/ml) |
|---|---|
| NO. | Mean |
| Example 7 | 93.58 |
| Example 8 | 181.73 |
| Example 9 | 243.42 |

It can be seen from the data in the above table that the preparation processes of Examples 8 and 9 significantly improved the oral plasma concentration of semaglutide tablets in beagle dogs.

### Example 10

1) Mixing: Prepare the drug preparation system powder by taking the raw materials. After the powder of each component is sieved through a 40-mesh sieve, the components are accurately weighed according to the mass preparation ratio shown below. After weighing material A, all of them are transferred into a PE bag, mixed 1-2 times, and shaken 100-200 times/time; after weighing material B, transfer them into a PE bag in order of mass from small to large, and shake 100-200 times for mixing each time a material is added.
Material A: 10.53% w/w semaglutide, 68.42% w/w microcrystalline cellulose, 21.05% w/w povidone K90
Material B: 11.38% w/w arginine, 34.12% w/w SNAC, 34.12% w/w sodium caprate, 17.24% w/w microcrystalline cellulose, 1.05% w/w sodium dodecyl sulfate, 2.09% w/w sodium carboxymethyl starch
2) Granulation: The dry granulator is set to set the cooling water temperature to 10-25° C., the roller pressure to 50±30 bar, the feeding speed to 10-30 rpm, the roller speed to 5-10 rpm, the granulation speed to 60-100 rpm, and the screen size to 16/20 mesh; the equipment is turned on to conduct trial granulation of material A. If it does not meet the requirements, the equipment parameters are appropriately adjusted and trial production is conducted again. If it meets the requirements, formal granulation is conducted. The actual parameters are recorded during the granulation process until all the powder is granulated to obtain granules A.

Granulate material B using the same process to obtain granules B.
3) Tabletting: According to the mass preparation ratio of 7.92% w/w granules A, 91.58% w/w granules B, and 0.5% w/w magnesium stearate, each component is accurately weighed, and after weighing, it is transferred into a PE bag, mixed 1-2 times, and shaken 100-200 times/time. After mixing, take the material for tableting. Use 13.5*7.5 mm shallow arc special-shaped punch for tableting, and feed 480 mg per tablet. Take the tablet out of the mold to get an oval tablet with 4 mg semaglutide.
4) Coating: Use Opadry® film coating premix to prepare a coating solution with a solid content of 15%, a coating gain of 4%-5%, and set the coating machine to an inlet temperature of 35° C., an outlet temperature of 30° C., an inlet speed of 1100-1300 rpm, an exhaust speed of 1300-1500 rpm, a main engine speed of 10-15 rpm, a spray pump speed of 2-10 rpm, an atomization pressure of 0.18 MPa, and a spray gun pressure of 0.2 MPa. It needs to be fully preheated before coating, and needs to be dried and solidified after coating.

### Example 11

1) Mixing: Prepare the drug preparation system powder by taking the raw materials. After the powder of each component is sieved through a 40-mesh sieve, the components are accurately weighed according to the mass preparation ratio shown below. After weighing material A, all of them are transferred into a PE bag, mixed 1-2 times, and shaken 100-200 times/time; after weighing material B, transfer them into a PE bag in order of mass from small to large, and shake 100-200 times for mixing each time a material is added.
Material A: 21.05% w/w semaglutide, 57.89% w/w microcrystalline cellulose, 21.05% w/w povidone K90
Material B: 11.38% w/w arginine, 34.12% w/w SNAC, 34.12% w/w sodium caprate, 17.24% w/w microcrystalline cellulose, 1.05% w/w sodium dodecyl sulfate, 2.09% w/w sodium carboxymethyl starch
2) Granulation: The dry granulator is set to set the cooling water temperature to 10-25° C., the roller pressure to 50±30 bar, the feeding speed to 10-30 rpm, the roller speed to 5-10 rpm, the granulation speed to 60-100 rpm, and the screen size to 16/20 mesh; the equipment is turned on to conduct trial granulation of material A. If it does not meet the requirements, the equipment parameters are appropriately adjusted and trial production is conducted again. If it meets the requirements, formal granulation is conducted. The actual parameters are recorded during the granulation process until all the powder is granulated to obtain granules A.

Granulate material B using the same process to obtain granules B.
3) Tabletting: According to the mass preparation ratio of 7.92% w/w granules A, 91.58% w/w granules B, and 0.5% w/w magnesium stearate, each component is accurately weighed, and after weighing, it is transferred into a PE bag, mixed 1-2 times, and shaken 100-200 times/time. After mixing, take the material for tableting. Use 13.5*7.5 mm shallow arc special-shaped punch for tableting, and feed 480 mg per tablet. Take the tablet out of the mold to get an oval tablet with 8 mg semaglutide.
4) Coating: Use Opadry® film coating premix to prepare a coating solution with a solid content of 15%, a coating gain of 4%-5%, and set the coating machine to an inlet temperature of 35° C., an outlet temperature of 30° C., an inlet speed of 1100-1300 rpm, an exhaust speed of 1300-1500 rpm, a main engine speed of 10-15 rpm, a spray pump speed of 2-10 rpm, an atomization pressure of 0.18 MPa, and a spray gun pressure of 0.2 MPa. It needs to be fully preheated before coating, and needs to be dried and solidified after coating.

### Example 12

1) Mixing: Prepare the drug preparation system powder by taking the raw materials. After the powder of each component is sieved through a 40-mesh sieve, the components are accurately weighed according to the mass preparation ratio shown below. After weighing material A, all of them are transferred into a PE bag, mixed 1-2 times, and shaken 100-200 times/time; after weighing material B, transfer them into a PE bag in order of mass from small to large, and shake 100-200 times for mixing each time a material is added.
Material A: 42.11% w/w semaglutide, 36.84% w/w microcrystalline cellulose, 21.05% w/w povidone K90
Material B: 11.38% w/w arginine, 34.12% w/w SNAC, 34.12% w/w sodium caprate, 17.24% w/w microcrystalline cellulose, 1.05% w/w sodium dodecyl sulfate, 2.09% w/w sodium carboxymethyl starch
2) Granulation: The dry granulator is set to set the cooling water temperature to 10-25° C., the roller pressure to 50±30 bar, the feeding speed to 10-30 rpm, the roller speed to 5-10 rpm, the granulation speed to 60-100 rpm, and the screen size to 16/20 mesh; the equipment is turned on to conduct trial granulation of material A. If it does not meet the requirements, the equipment parameters are appropriately adjusted and trial production is conducted again. If it meets the requirements, formal granulation is conducted. The actual parameters are recorded during the granulation process until all the powder is granulated to obtain granules A.

Granulate material B using the same process to obtain granules B.
3) Tabletting: According to the mass preparation ratio of 7.92% w/w granules A, 91.58% w/w granules B, and 0.5% w/w magnesium stearate, each component is accurately weighed, and after weighing, it is transferred into a PE bag, mixed 1-2 times, and shaken 100-200 times/time. After mixing, take the material for tableting. Use 13.5*7.5 mm shallow arc special-shaped punch for tableting, and feed 480 mg per tablet. Take the tablet out of the mold to get an oval tablet with 16 mg semaglutide.
4) Coating: Use Opadry® film coating premix to prepare a coating solution with a solid content of 15%, a coating gain of 4%-5%, and set the coating machine to an inlet temperature of 35° C., an outlet temperature of 30° C., an inlet speed of 1100-1300 rpm, an exhaust speed of 1300-1500 rpm, a main engine speed of 10-15 rpm, a spray pump speed of 2-10 rpm, an atomization pressure of 0.18 MPa, and a spray gun pressure of 0.2 MPa. It needs to be fully preheated before coating, and needs to be dried and solidified after coating.

### Test Example 4

The absorption of the semaglutide tablets prepared above after oral administration to cynomolgus monkeys was tested. The specific method is: the experimental animals need to fast for 12 hours and deprive of water for 6 hours before administration, and the fasting and deprivation of water are lifted 1 hour after administration. Before administration, a certain amount of blank blood is collected using an EDTA-K2 blood collection tube as the 0-point blood sample for the experiment and the animal weight is weighed. The tablets prepared in Examples 9-11 are orally administered to cynomolgus monkeys and 15 ml of water is given at the same time to ensure successful swallowing. Another group of semaglutide is administered intravenously to cynomolgus monkeys, and the dosage for each is 0.05 mg/kg. A total of 6 cynomolgus monkeys are used in each group, 3 females and 3 males. Subsequently, venous blood was collected using EDTA-K2 blood collection tubes at the following time points after administration: 0.25 h, 0.5 h, 1 h, 2 h, 4 h, 8 h, 24 h, 48 h, 72 h, 96 h, 120 h, 144 h, 168 h, 240 h, and 336 h. The whole blood of the cynomolgus monkeys was collected and centrifuged to obtain plasma samples. The samples were frozen at −20° C. and subsequently used for mass spectrometry analysis. The mass spectrometry detection was verified and implemented according to the method described in the public document “Quantitative Analysis of Semaglutide and Tirzepatide in Rat Plasma by SCIEX LC-MS/MS System”.

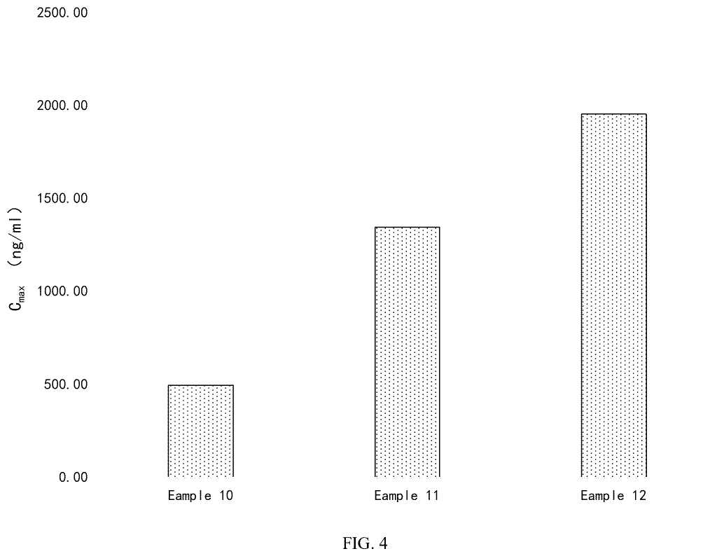

The absorption test results of the above-mentioned semaglutide tablets after oral administration in cynomolgus monkeys are shown in Table 5 and FIG. 4 below. The average bioavailability calculated after weight correction was 1.75%.

TABLE 5

| | Cₘₐₓ (ng/ml) |
|---|---|
| No. | Mean |
| Example 10 | 495.47 |
| Example 11 | 1344.83 |
| Example 12 | 1953.33 |

### Example 13, Preparation of Semaglutide Tablets

First, prepare the drug preparation system powder according to the scheme in Table 6. Specifically, weigh each component according to the mass configuration ratio shown in the table below, and mix after weighing.

TABLE 6
Semaglutide drug preparation system

| Pharmaceutical | |
|---|---|
| preparation | |
| system | Ingredients proportion |
| OLP-A001 | 1.52% semaglutide, 10.87% arginine, 32.61% |
| | SNAC, 32.61% sodium caprate, 22.39% |
| | microcrystalline cellulose |
| OLP-A003 | 1.52% semaglutide, 10.87% citrulline, 32.61% |
| | SNAC, 32.61% sodium caprate, 22.39% |
| | microcrystalline cellulose |
| OLP-A004 | 1.52% semaglutide, 10.87% niacinamide, 32.61% |
| | SNAC, 32.61% sodium caprate, 22.39% |
| | microcrystalline cellulose |
| OLP-A005 | 1.52% semaglutide, 65.22% SNAC, 33.26% |
| | microcrystalline cellulose |
| OLP-A006 | 1.52% semaglutide, 10.87% lysine, 21.74% SNAC, |
| | 43.48% sodium caprate, 22.39% microcrystalline |
| | cellulose |

A flat punch die was used for tableting, and the tablets were taken out from the die to obtain 10 mm round flat tablets with 7 mg semaglutide.

### Test Example 5, Beagle Dog Test on Semaglutide Tablets

Blank blood was collected before administration as the blood sample at 0 o'clock in the experiment. The tablets prepared in Example 13 and Novo Nordisk's original oral semaglutide tablets (Rybelsus®, 7 mg) were administered to beagle dogs by gavage, with 6 dogs in each group. Blood was then collected from the forearm vein of the beagle dogs at the following time points after administration: 0.5 h, 1 h, 2 h, and 3 h. The whole blood of the beagle dogs was collected and centrifuged, and the supernatant plasma blood samples were frozen after centrifugation for subsequent mass spectrometry analysis.

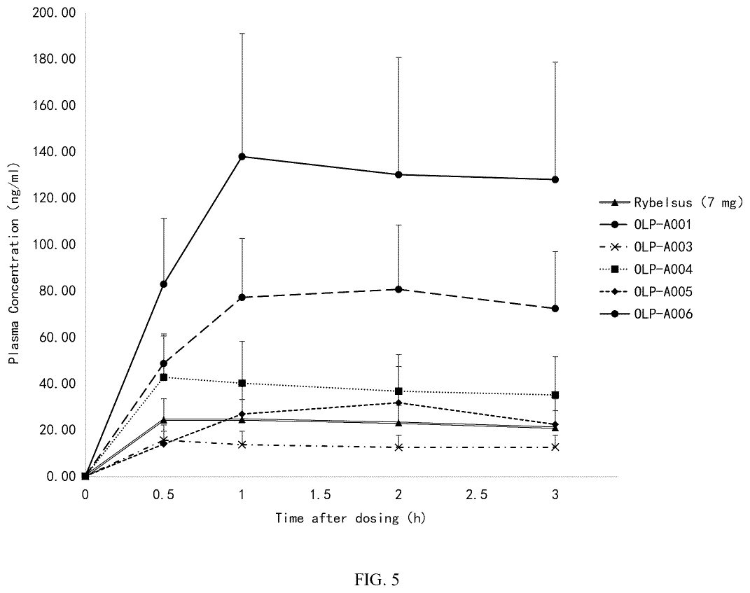

The experimental results are shown in Table 6-1. The experimental results showed that the absorption of semaglutide in the pharmaceutical preparation system OLP-A001 by beagle dogs was significantly better than that of Rybelsus® (FIG. 5).

TABLE 6-1
Analysis of the concentration of semaglutide in plasma after administration

| No. | Time after administration(h) | 0 | 0.5 | 1 | 2 | 3 |
|---|---|---|---|---|---|---|
| Rybelsus(7 mg) | Mean Plasma concentration(ng/ml) | 0.00 | 24.33 | 24.47 | 23.07 | 20.95 |
| | SEM | 0.00 | 9.21 | 8.63 | 7.56 | 7.31 |
| OLP-A001 | Mean Plasma concentration(ng/ml) | 0.00 | 48.62 | 77.12 | 80.58 | 72.32 |
| | SEM | 0.00 | 12.77 | 25.53 | 27.73 | 24.53 |
| OLP-A003 | Mean Plasma concentration(ng/ml) | 0.00 | 15.55 | 13.60 | 12.45 | 12.55 |
| | SEM | 0.00 | 6.54 | 5.82 | 5.33 | 5.26 |
| OLP-A004 | Mean Plasma concentration(ng/ml) | 0.00 | 42.67 | 40.10 | 36.65 | 35.05 |
| | SEM | 0.00 | 17.90 | 18.09 | 15.87 | 16.62 |
| OLP-A005 | Mean Plasma concentration(ng/ml) | 0.00 | 13.86 | 26.80 | 31.69 | 22.35 |
| | SEM | 0.00 | 5.63 | 12.79 | 15.68 | 11.17 |
| OLP-A006 | Mean Plasma concentration(ng/ml) | 0.00 | 82.79 | 137.83 | 130.04 | 127.92 |
| | SEM | 0.00 | 28.30 | 53.15 | 50.53 | 50.77 |

### Example 14, Preparation of Teriparatide Tablets

First, prepare the drug preparation system powder according to the scheme in Table 7. Specifically, weigh each component according to the mass configuration ratio shown in the table below, and mix after weighing.

TABLE 7
Teriparatide drug preparation system

| Pharmaceutical | |
|---|---|
| preparation | |
| system | Ingredients proportion |
| OLP-B001 | 1.52% teriparatide, 10.87% arginine, 32.61% SNAC, |
| | 32.61% sodium caprate, 22.39% microcrystalline |
| | cellulose |
| OLP-B002 | 1.52% teriparatide, 98.48% microcrystalline cellulose |
| OLP-B003 | 1.52% teriparatide, 65.22% SNAC, 33.26% |
| | microcrystalline cellulose |

### Test Example 6, Beagle Dog Test on Teriparatide Tablets

Before administration, 1 ml of blank blood was collected as the blood sample at 0 o'clock of the experiment. The tablets prepared in Example 14 were administered to beagle dogs. Subsequently, blood was collected from the forearm vein of the beagle dogs at the following time points after administration: 0.25 h, 0.5 h, 1 h, 2 h, and 3 h. The whole blood of the beagle dogs was collected and centrifuged, and the supernatant plasma blood samples were frozen after centrifugation for subsequent mass spectrometry analysis.

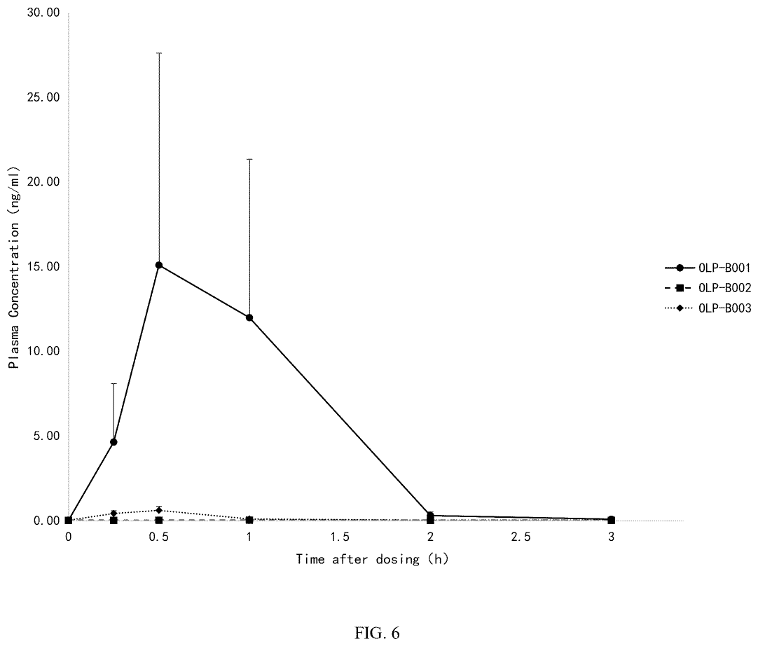

The experimental results are shown in Table 7-1. The experimental results showed that the absorption of teriparatide in the pharmaceutical preparation system OLP-1B001 by beagle dogs was significantly better than that of OLP-1B003 (FIG. 6).

TABLE 7-1
Analysis of the concentration of teriparatide in plasma after administration

| No. | Time after administration(h) | 0 | 0.25 | 0.5 | 1 | 2 | 3 |
|---|---|---|---|---|---|---|---|
| OLP-B001 | Mean Plasma concentration(ng/ml) | 0.00 | 4.62 | 15.08 | 11.97 | 0.28 | 0.07 |
| | SEM | 0.00 | 3.45 | 12.53 | 9.36 | 0.24 | 0.03 |
| OLP-B002 | Mean Plasma concentration(ng/ml) | 0.00 | 0.00 | 0.01 | 0.01 | 0.00 | 0.01 |
| | SEM | 0.00 | 0.00 | 0.01 | 0.01 | 0.00 | 0.01 |
| OLP-B003 | Mean Plasma concentration(ng/ml) | 0.00 | 0.41 | 0.59 | 0.08 | 0.01 | 0.00 |
| | SEM | 0.00 | 0.15 | 0.26 | 0.03 | 0.01 | 0.00 |

### Example 15, Preparation of Tirzepatide Capsules

First, prepare the drug preparation system powder according to the scheme in Table 8. Specifically, weigh each component according to the mass configuration ratio shown in the table below, and mix after weighing.

TABLE 8
Tirzepatide drug preparation system

| Pharmaceutical | |
|---|---|
| preparation | |
| system | Ingredients proportion |
| OLP-C001 | 3.04% tirzepatide e, 10.87% arginine, 32.61% |
| | SNAC, 32.61% sodium caprate, 20.87% |
| | microcrystalline cellulose |
| OLP-C002 | 3.04% tipeptide, 96.96% microcrystalline cellulose |
| OLP-C003 | 3.04% tirzepatide, 65.22% SNAC, 31.74% |
| | microcrystalline cellulose |
| OLP-C004 | 3.04% tirzepatide, 10.87% niaconamide, 32.61% |
| | SNAC, 32.61% sodium caprate, 20.87% |
| | microcrystalline cellulose |
| OLP-C005 | 3.04% tirzepatide, 10.87% citrulline, 32.61% |
| | SNAC, 32.61% sodium caprate, 20.87% |
| | microcrystalline cellulose |

The tablets were pressed using a flat punch die, and the tablets were taken out of the die to obtain 8 mm round flat tablets with 4.86 mg tirzepatide. The above tablets were ground and passed through an 80-mesh sieve, and 13.16 mg of the sieved powder was weighed and filled into a No. 9 gelatin capsule to obtain a No. 9 capsule with 0.4 mg tirzepatide.

### Test Example 7, Trial on Tirzepatide Capsules in Rats

Before administration, blank blood was collected from rats as the blood sample at zero point of the experiment. After the rats were anesthetized, the abdomen was shaved and the laparotomy was performed. The small intestine about 12 cm away from the rat's cecum was carefully cut open with a blade and the rat's intestine was given a capsule prepared in Example 15 through the incision. The capsule was pushed 2 cm toward the cecum to avoid the wound, and then the rat's intestine and abdomen were sutured. Blood was collected from rats at the following time points after administration: 0.5 h, 1 h, 2 h, 3 h, and 4 h, and the blood volume at each blood sampling point was 0.5 ml. The whole blood of the rats was collected and centrifuged, and the supernatant plasma blood samples were frozen after centrifugation for subsequent mass spectrometry analysis.

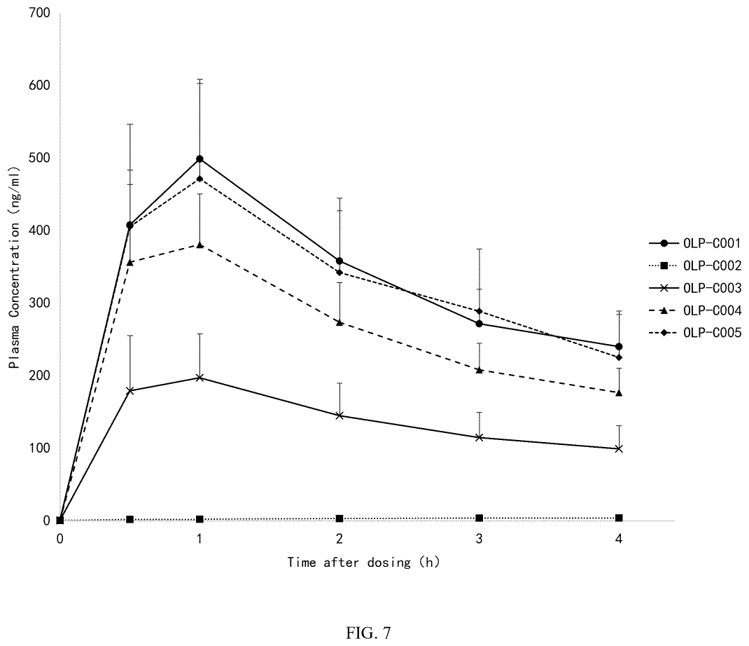

The experimental results are shown in Table 8-1. The experimental results showed that rats absorbed Tirzepatide in the pharmaceutical preparation systems OLP-C001 and OLP-C005 better than OLP-C003 and OLP-C004 (FIG. 7).

TABLE 8-1
Analysis of the concentration of tirzepatide in plasma after administration

| No | Time after administration (h) | 0 | 0.5 | 1 | 2 | 3 | 4 |
|---|---|---|---|---|---|---|---|
| OLP-C001 | Mean Plasma concentration (ng/ml) | 0.00 | 407.08 | 498.12 | 357.46 | 271.00 | 239.47 |
| | SEM | 0.00 | 75.90 | 104.26 | 69.28 | 47.52 | 44.59 |
| OLP-C002 | Mean Plasma concentration (ng/ml) | 0.00 | 1.29 | 1.67 | 2.60 | 3.20 | 3.51 |
| | SEM | 0.00 | 0.59 | 0.42 | 0.59 | 0.72 | 0.93 |
| OLP-C003 | Mean Plasma concentration (ng/ml) | 0.00 | 178.51 | 196.48 | 144.33 | 114.12 | 98.56 |
| | SEM | 0.00 | 76.37 | 60.73 | 45.06 | 35.00 | 32.01 |
| OLP-C004 | Mean Plasma concentration (ng/ml) | 0.00 | 355.70 | 380.05 | 273.00 | 207.46 | 176.03 |
| | SEM | 0.00 | 107.32 | 69.97 | 54.80 | 36.60 | 34.11 |
| OLP-C005 | Mean Plasma concentration (ng/ml) | 0.00 | 404.62 | 470.76 | 341.52 | 288.17 | 224.52 |
| | SEM | 0.00 | 141.37 | 137.37 | 102.80 | 85.92 | 64.35 |

### Example 16, Preparation of Octreotide Capsules

First, prepare the drug preparation system powder according to the scheme in Table 9. Specifically, weigh each component according to the mass configuration ratio shown in the table below, and mix after weighing.

TABLE 9
Octreotide pharmaceutical preparation system

| Pharmaceutical | |
|---|---|
| preparation | |
| system | Ingredients proportion |
| OLP-D001 | 3.04% Octreotide, 10.87% arginine, 32.61% |
| | SNAC, 32.61% sodium caprate, 22.39% |
| | microcrystalline cellulose |
| OLP-D002 | 3.04% Octreotide, 96.96% microcrystalline cellulose |
| OLP-D003 | 3.04% Octreotide, 65.22% SNAC, 31.74% |
| | microcrystalline cellulose |
| OLP-D004 | 3.04% Octreotide, 10.87% citrulline, 32.61% |
| | SNAC, 32.61% sodium caprate, 22.39% |
| | microcrystalline Cellulose |

The experimental results are shown in Table 9-1. he tablets were pressed using a flat punch die, and the tablets were taken out of the die to obtain 8 mm round flat tablets with 4.86 mg octreotide. The above tablets were ground and passed through an 80-mesh sieve, and 13.16 mg of the sieved powder was weighed and filled into a No. 9 gelatin capsule to obtain a No. 9 capsule with 0.4 mg octreotide.

### Test Example 8, Octreotide Capsule Rat Test

Before administration, blank blood was collected from rats as the blood sample at 0 point of the experiment. After the rats were anesthetized, the abdomen was shaved and the laparotomy was performed. Then, the small intestine about 12 cm away from the rat's cecum was carefully cut open with a blade and the rat's intestine was given a capsule described in Example 16 through the incision. The capsule was pushed 2 cm toward the cecum to avoid the wound, and then the rat's intestine and abdomen were sutured. Blood was collected from rats at the following time points after administration: 0.5 h, 1 h, 2 h, 3 h, and 4 h, and the blood volume at each blood sampling point was 0.5 ml. The whole blood of the rats was collected and centrifuged. After centrifugation, the supernatant serum blood samples were frozen and subsequently used for mass spectrometry analysis.

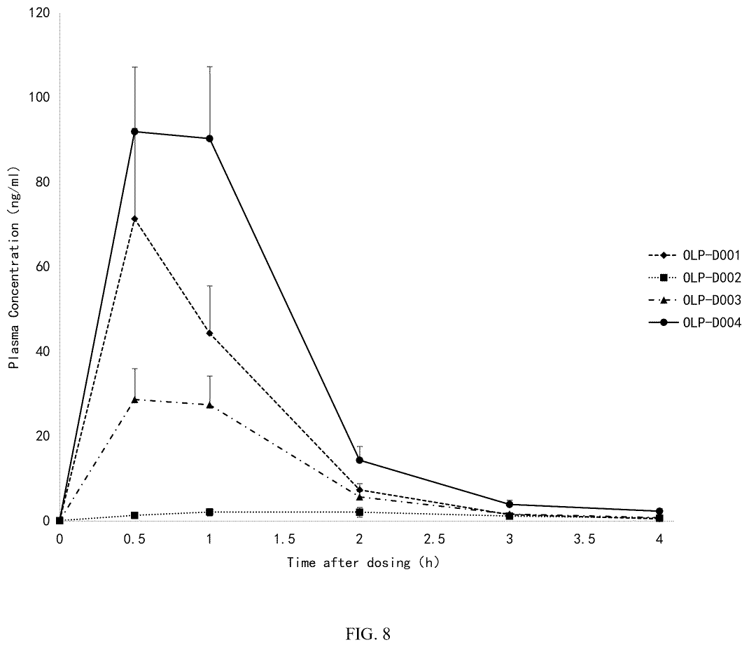

The experimental results are shown in Table 9-1. The experimental results showed that the absorption of octreotide in the Pharmaceutical formulation system OLP-1D001 and OLP-D004 by rats was significantly better than that of OLP-D003 (FIG. 8).

TABLE 9-1
Analysis of octreotide concentration in plasma after administration

| No. | Time after administration (h) | 0 | 0.5 | 1 | 2 | 3 | 4 |
|---|---|---|---|---|---|---|---|
| OLP-D001 | Mean Plasma concentration (ng/ml) | 0.00 | 71.27 | 44.25 | 7.26 | 1.41 | 0.42 |
| | SEM | 0.00 | 21.39 | 11.16 | 1.46 | 0.31 | 0.15 |
| OLP-D002 | Mean Plasma concentration (ng/ml) | 0.00 | 1.25 | 2.02 | 2.00 | 1.07 | 0.58 |
| | SEM | 0.00 | 0.27 | 0.87 | 1.11 | 0.59 | 0.39 |
| OLP-D003 | Mean Plasma concentration (ng/ml) | 0.00 | 28.60 | 27.34 | 5.60 | 1.55 | 0.69 |
| | SEM | 0.00 | 7.33 | 6.79 | 1.36 | 0.35 | 0.22 |
| OLP-D004 | Mean Plasma concentration (ng/ml) | 0.00 | 91.84 | 90.21 | 14.27 | 3.83 | 2.22 |
| | SEM | 0.00 | 15.24 | 16.98 | 3.23 | 1.01 | 0.63 |

### Test Example 9, Characterization of Composition

Weigh the powder according to the scheme in Table 9, add PBS and vortex. After vortexing, centrifuge the sample and take the supernatant into the sample pool, and analyze it using a particle size analyzer.

TABLE 9
pharmaceutical preparation system

| Pharmaceutical | |
|---|---|
| preparation | |
| system | Ingredients proportion |
| OLP-S001 | 10 mg semaglutide |
| OLP-S002 | 10 mg semaglutide, 37.5 mg arginine, 112.5 mg sodium |
| | caprate, 112.5 mg SNAC |
| OLP-S003 | 10 mg semaglutide, 225 mg SNAC |

The experimental results show that the peptides in the drug system OLP-S002 can form micelles with a mean particle size of 2.84.

TABLE 10
Peak mean value of the combined drug particle size distribution

| Pharmaceutical | Particle size distribution |
|---|---|
| preparation system | peak mean (nm) ± SD |
| OLP-S001 | 4.48 ± 0.09 |
| OLP-S002 | 2.84 ± 0.51 |
| OLP-S003 | 697.13 ± 67.67 |

What is claimed is:

1. An oral polypeptide composition, wherein the oral polypeptide composition comprises: a polypeptide targeting the GLP-1 receptor, one or more amino acids present as individual components, N-(8-[2-hydroxybenzoyl]-amino) caprylic acid (NAC) or a salt thereof, and medium-chain fatty acids or a salt thereof; a total content of the one or more amino acids, the NAC or salt thereof, and the medium-chain fatty acid or a salt thereof in the oral polypeptide composition is 50-90% w/w.

2. The oral polypeptide composition according to claim 1, wherein the oral polypeptide composition comprises no more than 20% w/w of the one or more amino acids.

3. The oral polypeptide composition according to claim 1, wherein the oral polypeptide composition comprises no more than 55% w/w of the NAC or a salt thereof.

4. The oral polypeptide composition according to claim 1, wherein the oral polypeptide composition comprises no less than 10% w/w of the medium-chain fatty acid or a salt thereof.

5. The oral polypeptide composition according to claim 1, wherein the oral polypeptide composition comprises no more than 15% w/w of the polypeptide targeting the GLP-1 receptor.

6. The oral polypeptide composition according to claim 1, wherein a mass ratio of the one or more amino acids, the NAC or salt thereof, and the medium-chain fatty acid or a salt thereof is 1:(2.5-3.5):(2.5-3.5).

7. The oral polypeptide composition according to claim 1, wherein the polypeptide is semaglutide.

8. The oral polypeptide composition according to claim 1, wherein the oral polypeptide composition comprises: semaglutide, about 10-20% w/w of the one or more amino acids, 20-50% of the NAC or the salt thereof, and no less than 15% w/w of the medium-chain fatty acid or a salt thereof; wherein the total content of the one or more amino acids, the NAC or salt thereof, and the medium-chain fatty acid or a salt thereof in the oral polypeptide composition is 70-80% w/w.

9. The oral polypeptide composition according to claim 1, wherein the salt of the medium-chain fatty acid is sodium caprate.

10. The oral polypeptide composition according to claim 1, wherein the one or more amino acids comprising nitrogen in a side chain are selected from lysine, arginine, asparagine, glutamine, tryptophan, ornithine, citrulline, pyrrolysine, or histidine.

11. The oral polypeptide composition according to claim 1, wherein the salt of the NAC is sodium N-(8-(2-hydroxybenzoyl) amino) caprylate (SNAC).

12. The oral polypeptide composition according to claim 1, wherein the oral polypeptide composition further comprises no more than 30% w/w of excipients.

13. The oral polypeptide composition according to claim 1, wherein the oral polypeptide composition comprises:
1-50 pbw of semaglutide;
15-100 pbw of arginine;
100-300 pbw of sodium caprate;
100-300 pbw of SNAC; and
40-180 pbw of excipients.

14. The oral polypeptide composition according to claim 1, wherein the oral polypeptide composition comprises: 4.0 mg semaglutide, 50.0 mg arginine, 150.0 mg SNAC, 150.0 mg sodium caprate, and 126.0 mg excipients.

15. The oral polypeptide composition according to claim 1, wherein the oral polypeptide composition comprises: 8.0 mg semaglutide, 50.0 mg arginine, 150.0 mg SNAC, 150.0 mg sodium caprate, and 122.0 mg excipients.

16. The oral polypeptide composition according to claim 1, wherein the oral polypeptide composition comprises: 16.0 mg semaglutide, 50.0 mg arginine, 150.0 mg SNAC, 150.0 mg sodium caprate, and 114.0 mg excipients.

17. The oral polypeptide composition according to claim 1, wherein the oral polypeptide composition is a solid preparation.

18. The oral polypeptide composition according to claim 17, wherein the oral polypeptide composition is tablets, capsules, granules, powders, granules, pellets, or pills.

19. The oral polypeptide composition according to claim 18, wherein a specification of the tablets is 1-50 mg.

20. A method for preventing and/or treating a disease, comprising administering the oral polypeptide composition according to claim 1 to a subject in need; wherein the disease is selected from at least one of type 1 diabetes, type 2 diabetes, obesity, cardiovascular disease, non-alcoholic fatty liver disease, chronic kidney disease, Alzheimer's disease, cancer, polycystic ovary syndrome, metabolic syndrome, bone metabolism and osteoporosis, knee osteoarthritis, sleep apnea syndrome, osteoarthritis, and metabolic muscle disease.
